(19) United States
(12) Patent Application Publication
(10) Pub. No.: US 2009/0156563 A1
(43) Pub. Date: Jun. 18, 2009

(54) Glucan Compositions

(76) Inventors: Werner Baschong, Basel (CH); Sebastien Mongiat, Sierentz (FR); Dietmar Ochs, Schopfheim (DE)

Correspondence Address: JoAnn Villamizar;Ciba Corporation/Patent Department, 540 White Plains Road, P.O. Box 2005, Tarrytown, NY, 10591, US

(21) Appl. No.: 12/085,271

(22) PCT Filed: Nov. 20, 2006
(86) PCT No.: PCT/EP2006/068645

§ 371 Date: May 20, 2008

(30) Foreign Application Priority Data
Nov. 30, 2005 (EP) 05111504.6
Jul. 7, 2006 (EP) 06116766.4

(51) Int. Cl. A61K 31/60; A61P 17/00

(52) U.S. Cl. 514/159

(57) ABSTRACT

A cosmetic or pharmaceutical composition is provided comprising: a) 0.001 to less than 0.2% by weight, based on the weight of the total composition, of a scleroglucan having a mean molecular weight of 1×10⁶ to 12×10⁶; b) a cosmetically or pharmaceutically acceptable carrier; and, optionally, c) lactic acid, a lactate and/or pentanediol. The composition shows advantageous effects, such as moisturization of skin or mucosa, and has an antiaging and revitalizing effect on the skin. When combined with a certain bactericide as explained in claim 1, compositions containing native polysaccharides of the glucan class in general, especially of the scleroglucan as of component a), are well suitable to reduce the number of bacteria in mucosal or oral environments and minimise adhesion, while retaining a pleasant feeling on the mucosa or gingiva. Plaque thus can be efficiently prevented or removed.

The present invention relates to compositions useful in the field of cosmetics and pharmaceuticals, especially skin cosmetics and dermatological compositions, containing a low concentration of a specific polysaccharide of the scleroglucan class, and optional further components like lactate or pentanediol. The present invention further relates to antibacterial compositions for contact with the mucosa such as an oral care or feminine hygiene composition, which contain synergistic mixture of a glucan, especially the above scleroglucan, and a specific bactericide.

EP-A-655904 recommends 1,2-pentanediol as a skin moisturizer.

U.S. Pat. No. 5,814,341 describes the use of β-1,3-scleroglucans for the preparation of stable and transparent gels. In U.S. Pat. No. 6,162,447 and U.S. Pat. No. 6,162,449, the preparation of such polysaccharides by cultivation of microorganisms is explained, and personal care formulations, including microemulsions, containing these scleroglucans are disclosed, inter alia effecting skin lubrication, moisturization, film formation and improvement of skin sensory properties, and/or acting as dispersion aid or (co)emulsifier, thickening agent, retention aid for other active ingredients. High amounts of scleroglucan are generally used according to these documents, especially in skin applications.

JP-A-2001031541 and WO 99/32073 teach the use of a native polysaccharide such as a β-D-glucan or a modified polysaccharide such as a phosphochitosan in an oral composition, e.g. for the prevention and control of tooth tartar, dental plaque, or periodontitis.

These dental affections, like caries or a group of mucosal diseases, are largely caused by the attack of microorganisms such as bacteriae. Their harmful action is often enhanced by their ability to adhere to their substrate, forming a biofilm especially on hard tissue or hard tissue/soft tissue interfaces. Further they may produce harmful enzymes or other agents such as alkaline phosphatase, and by rapid proliferation.

A combination of a certain glucosamin with chlorohexidine has been reported to strongly reduce infestation of oral environments with bacteriae (see Decker et al., J. Peridont Res. 40, 373 (2005)).

It has now been found, that the amount of scleroglucan used in formulation applied to or in contact with the skin surprisingly may be greatly reduced while advantageous effects are retained or even improved, especially with concomitant use of a further component as explained below. It has further been found that this use plays an antiaging and revitalizing effect on the skin.

Subject of the Invention Thus is a Composition Comprising

a) 0.001 to 0.2%, or less than 0.2%, relative to the weight of the total composition, of a scleroglucan of mean molecular weight 1·10⁶ to 12·10⁶;
b) a cosmetically or pharmaceutically acceptable carrier; and
c) a further component selected from lactic acid, lactate and 1,2-pentanediol.

In the cosmetic or pharmaceutic end product, component a) is mainly used in a concentration from 0.001 to 0.1%, especially 0.005 to less than 0.05% by weight, relative to the weight of the total product composition.

The lactate often is sodium lactate, preferred as component c) thus are sodium lactate and/or 1,2-pentanediol. Component c) is preferably used in an amount of 0.005 to 3, especially 0.02 to 1.0% by weight, relative to the weight of the total product composition.

Specifically, the weight ratio used of component (c) relative to component (a) (c:a) often is from the range 1:1 to 30:1, for example 2:1 to 20:1, especially about 2-12 or 5-10 parts by weight of lactic acid or a lactate such as sodium lactate and/or 5-20, especially 8 to 18 parts by weight of 1,2-pentanediol, on one part by dry weight of the scleroglucan in component (a).

Polysaccharides of the scleroglucan class useful in the present invention are known e.g. from U.S. Pat. No. 5,814,341, U.S. Pat. No. 6,162,447 and U.S. Pat. No. 6,162,449. The scleroglucan to be used in the present invention is to be understood as a product which may be isolated from the fungi Sclerotium, Lentillium or Schizophyllium (thus also called scleroglucan, lentinan, schizophyllan) and may be been obtained by cultivation of microorganisms in a manner as described in the US patents cited above, preferably microorganisms in the form of the plant-pathogenic fungi imperfecti Sclerotium rolfsii.

Scleroglucanes of the present class are also recalled as β-1,3(1,6)-glucanes or β-1,3-scleroglucanes. The polysaccharide chains usually form a three-dimensional structure of triple helices; polymer chains essentially consist of glucose units whose hydroxy groups in 1- and 3-position are β-linked to form the polymer main chain, and wherein each 3^{rd} glucose unit contains in position 6 a further glucose moiety linked by its 1-OH funktion (β-1,3-bonded glucopyranose as the main chain and β-1,6-bonded glucopyranose as side chains) and has the structural formula:

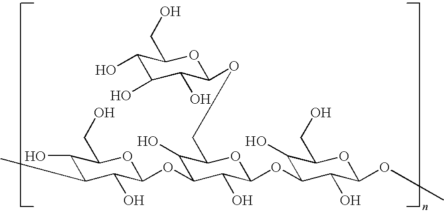

in which n is a number which provides the β-1,3-scleroglucan component with a mean molecular weight (MW) of 1×06 to 12×10⁶, preferably 2×10⁶ to 10×10⁶. All molecular weights (MW) noted are determined from the readily measured Staudinger Index η using the following Mark-Houwink equation:

MW=[η/4×45×10⁻⁷]^{1/1.49}.

Preferably, a 0.3 g/l aqueous solution of the β-1,3-scleroglucan has a glucose content below 0.1 g/l and a viscosity of 50 to 190 mPa·s, measured at a shear rate of 0.3 s⁻¹ at 20° C.

Preferred is the β-1,3-scleroglucan produced using the plant-pathogenic fungi imperfecti Sclerotium rolfsii ATCC 15205 (U.S. Pat. No. 6,162,447 and U.S. Pat. No. 6,162,449).

Though of relatively high molecular weight, the present glucane component, especially the scleroglucanes described above, are soluble in aqueous solutions. In consequence, essentially no increase in turbidity is caused by addition of the present glucan component. Compositions of the invention are mainly useful in the field of cosmetics and pharmaceuticals especially for the skin treatment, e.g. as cosmetical or dermatological skin composition.

It has been found that the combination of the scleroglucan with a lactate such as sodium lactate, and/or with 1,2-pentanediol in the formulation acts in an especially advantageous manner as a moisturizer or lubricant on the skin or mucosa. Formulations containing the combination of the scleroglucan with lactate show further advantageous effects including improved stability of the formulation under high- or low temperature storage conditions (e.g. at 0-10° C.) and improved resistance against microbial attack.

The composition of the invention preferably is in the form of a gel or viscous liquid (e.g. of viscosity range 2-100000 cp, especially 10-20000 cp at 20° C. (cp=centipoise)).

It has further been found that the present scleroglucan formulation plays an antiaging effect or contributes to such an effect, e.g. by
improving the appearance of the skin and helping in the wound healing,
reduction of wrinkle depth and height; and plays a revitalizing effect on the skin's active defense system (skin immune system) by
protecting against the damaging effects of UV radiation and photooxidation, especially on the Langerhans cells,
activating the Langerhans cells, and
promoting the growth of keratinocytes.

As has now been found, extremely low amounts of the scleroglucan may be used, while its advantageous effects are retained or even improved; especially worth emphasizing are the high degree of moisturization, and skin effects such as reduced stickiness, reduced roughness, improved softness, general skin health.

A further aspect of the invention thus is a composition, especially a cosmetical or dermatological skin composition, comprising 0.001 to less than 0.05% by weight, especially 0.005 to less than 0.05% by weight, relative to the weight of the total composition, of a β-1,3-scleroglucan of mean molecular weight 1·10⁶ to 12·10⁶, and a cosmetically or pharmaceutically acceptable carrier.

It has been a further finding of the invention, that native polysaccharides of the glucan class are especially well suitable to reduce the number of bacteria in mucosal or oral environments and minimise adhesion, when combined with a suitable bactericide, while retaining a pleasant feeling on the mucosa or gingiva. Plaque can be efficiently prevented or removed.

Subject of the invention thus is an antibacterial composition for contact with the mucosa and other tissues of the oral cavity, which is characterized by containing

a) a glucan and
b) a bactericide selected from benzoic acid, its salts and esters; propionic acid and its salts; salicylic acid and its salts; sorbic acid and its salts; formaldehyde; paraformaldehyde; o-phenylphenol and its salts; inorganic sulphites and hydrogen sulphites; sodium iodate; chlorobutanol; 4-hydroxybenzoic acid and its salts and esters; 3-acetyl-6-methylpyran-2,4(3H)-dione; formic acid; sodium formiate; dibromohexamidine and its salts; undec-10-enoic acid and salts; hexetidine; 5-bromo-5-nitro-1,3-dioxane; bronopol; 2,4-dichlorobenzyl alcohol; triclocarban; Triclosan; 4-chloro-3,5-xylenol; imidazolidinyl urea; poly(1-hexamethylenebiguanide hydrochloride); 2-phenoxyethanol; hexamethylenetetramine; methenamine 3-chloroallylochloride; 1-(4-chlorophenoxy)-1-(imidazol-1-yl)-3,3-dimethylbutan-2-one; 1,3-bis(hydroxymethyl)-5,5-dimethylimidazolidine-2,4-dione; benzyl alcohol; 1-hydroxy-4-methyl-6(2,4,4-trimethylpentyl)-2-pyridon or its monoethanolamine salt; methyldibromoglutaronitrile; bromochlorophen; 4-isopropyl-m-cresol; mixture of 5-Chloro-2-methyl-isothiazol-3(2H)-one and 2-methylisothiazol-3(2H)-one with magnesium chloride and magnesium nitrate; clorophene; 2-chloroacetamide; chlorhexidine and its digluconate, diacetate and/or dihydrochloride; 1-phenoxypropan-2-ol; alkyl (C₁₂-C₂₂) trimethyl ammonium bromide and/or chloride; 4,4-dimethyl-1,3-oxizalidine; N-(hydroxymethyl)-N-(dihydroxymethyl-1,3-dioxo-2,5-imidazolidinyl-4)-N′-(hydroxymethyl)urea; hexamidine and its salts; glutaraldehyde; chlorphenesin; sodium hydroxymethylglycinate; benzethonium chloride; benzalkonium chloride, bromide and/or saccharinate; benzylhemiformal, listerine, alexidine. Chlorhexidine may be in free form or any of its application forms such as the gluconate or acetate or hydrochloride.

Salts and other derivatives such as esters in the following, more detailed list of bactericides useful as component b), are to be understood as cosmetically or pharmacologically, depending on the intended end-use, acceptable salts and derivatives (percentages are preferred amounts, given by weight relative to the total composition; most preferred is a dosage within the range of about 50% of the upper limit and the upper limit of range given):

Benzoic acid, its salts and esters 0.01-0.05, especially 0.05-0.5% b.w. of acid;
Propionic acid and its salts 0.01-2% b.w. of acid;
Salicylic acid and its salts 0.01-0.5% b.w. of acid;
Sorbic acid (hexa-2,4-dienoic acid) and its salts 0.01-0.6% b.w. of acid;
Formaldehyde or paraformaldehyde 0.01-0.2% (not for oral compositions) or 0.01-0.1% (oral compositions);
Biphenyl-2-ol (o-phenylphenol) and its salts 0.01-0.2%;
Inorganic sulphites and hydrogen sulphites 0.01-0.2% b.w. of free SO₂;
Sodium iodate 0.01-0.1%;

### Chlorobutanol (INN) 0.01-0.5%;

4-Hydroxybenzoic acid and its salts and esters 0.01-4% b.w. of acid, especially 0.01-0.8% b.w. of acid, e.g. when using a mixture of esters;
3-Acetyl-6-methylpyran-2,4(3H)-dione (Dehydracetic acid) and its salts 0.01-0.6% b.w. of acid;
Formic acid and its sodium salt 0.01-0.5% b.w. of acid;
3,3′-Dibromo-4,4′-hexamethylenedioxydibenzamidine (Dibromohexamidine) and its salts (including isethionate) 0.01-0.1%;
Undec-10-enoic acid and salts 0.01-0.2% b.w. of acid;

### Hexetidine (INN) 0.01-0.1%;

5-Bromo-5-nitro-1,3-dioxane 0.01-0.1%;

### Bronopol (INN) 0.01-0.1%:

2,4-Dichlorobenzyl alcohol 0.01-0.15%;

### Triclocarban (INN) 0.01-0.2%;

### Triclosan (INN) 0.01-0.3%;

4-Chloro-3,5-xylenol 0.01-0.5%;
3,3′-Bis (1-hydroxymethyl-2,5-dioxoimidazolidin-4-yl)-1,1′-methylenediurea (Imidazolidinyl urea) 0.01-0.6%;
Poly (1-hexamethylenebiguanide hydrochloride 0.01-0.3%;

### 2-Phenoxyethanol 0.01-1,0%;

Hexamethylenetetramine (methenamine) (INN) 0.01-0.15%;
Methenamine 3-chloroallylochloride (INNM) 0.01-0.2%;
1-(4-Chlorophenoxy)-1-(imidazol-1-yl)-3,3-dimethylbutan-2-one 0.01-0.5%;
1,3-Bis (hydroxymethyl)-5,5-dimethylimidazolidine-2,4-dione 0.01-0.6%;
Benzyl alcohol 0.01-1.0%;
1-Hydroxy-4-methyl-6(2,4,4-trimethylpentyl) 2-pyridon or its monoethanolamine salt 0.01-1%;
1,2-Dibromo-2,4-dicyanobutane (methyldibromoglutaronitrile) 0.01-0.1%;
6,6-Dibromo-4,4-dichloro-2,2′-methylenediphenol (Bromochlorophen) 0.01-0.1%;

### 4-Isopropyl-m-cresol 0.01-0.1%;

Mixture of 5-Chloro-2-methyl-isothiazol-3(2H)-one and 2-methylisothiazol-3(2H)-one (especially in the ratio 3:1) with magnesium chloride and magnesium nitrate 0.0005-0.0015%;
2-Benzyl-4-chlorophenol (clorophene) 0.01-0.2%;

### 2-Chloroacetamide 0.01-0.3%;

Chlorhexidine (INN) and its digluconate, diacetate and/or dihydrochloride 0.01-0.3% b.w. of chlorhexidine;

### 1-Phenoxypropan-2-ol 0.01-1.0%;

Alkyl (C12-C22) trimethyl ammonium, bromide and/or chloride 0.01-0.1%;
4,4-dimethyl-1,3-oxizalidine 0.01-0.1% (in finished product of pH 6 or higher);
N-(Hydroxymethyl)-N-(dihydroxymethyl-1,3-dioxo-2,5-imidazolidinyl-4)-N′-(hydroxymethyl)urea 0.01-0.5%;
1,6-Di (4-amidinophenoxy)-n-hexane (Hexamidine) and its salts (including isethionate and p-hydroxybenzoate) 0.01-0.1%;
Glutaraldehyde (Pentane-1,5-dial) 0.01-0.1%;
3-(p-chlorophenoxy)-propane-1,2 diol (chlorphenesin) 0.01-0.3%;
Sodium hydroxymethylamino acetate (Sodium Hydroxymethylglycinate) 0.01-0.5% Benzethonium chloride 0.01-0.1%;
Benzalkonium chloride, bromide and/or saccharinate 0.01-0.1% (b.w. calculated as benzalkonium chloride; not for eye care formulations);

### Benzylhemiformal 0.01-0.15%;

Listerine (5-methyl-2-(1-methylethyl)-phenol mixed with 5-methyl-2-(1-methylethyl)cyclohexanol and 1,3,3-trimethyl-2-oxabicyclo(2.2.2)octane) 0.01-0.5%.

Preferred bactericides of component b) are selected from 5-chlor-2-(2,4-dichlorophenoxy)-phenol (Triclosan), alexidine, hexetidine, benzalkonium chloride, salicylamide, domiphen bromide, tetradecylpyridinium chloride, N-tetradecyl-4-ethylpyridinium chloride, octenifine, delmopinol, octapinol and other piperidine derivatives, zinc/stannous ion agents; essential oils including thymol, geraniol, carvacrol, citral, hinokitiol, eucalyptol, eugenol, menthol, catechol; and mixtures thereof.

Also important are bactericides selected from 2-phenylphenol, 2.4.4′-trichloro-2′-hydroxy-diphenylether (Triclosan), 4,4′-dichloro-2-hydroxydiphenylether, 2,2′-methylene-bis-(4-chloro-phenol), 4-(2-t-butyl-5-methylphenoxy)-phenol, 3-(4-chlorophenyl)-1-(3,4-dichloro-phenyl)-urea, chlorhexidine in free form or any of its application forms such as the gluconate or acetate or hydrochloride, hexetidine, benzalkonium chloride. Most important are Triclosan, chlorohexidin, hexetidine, listerine.

Essential oils are often added as additional components besides one of the other bactericides mentioned, especially in oral care compositions.

Of special importance is an oral care composition containing as component b) an anti-microbial agent selected from 5-chlor-2-(2,4-dichlorophenoxy)-phenol (Triclosan), Silver Dihydrogen Citrate, Phthalic acid and its salts, alexidine, hexetidine, sanguinarine, benzalkonium chloride, salicylamilide, domiphen bromide, cetylpyridinium chloride, tetradecylpyridinium chloride, N-tetradecyl-4-ethylpyridinium chloride, octenifine, delmopinol, octapinol and other piperidine derivatives, nicin preparations, zinc/stannous ion agents, essential oils including thymol, geraniol, carvacrol, citral, hinokitiol, eucalyptol, catechol and mixtures thereof.

The composition contains the glucan usually in an amount of 0.001 to 0.2%, such as 0.001 to 0.1, especially 0.005 to less than 0.05% by weight, relative to the weight of the total composition. The preferred glucan is a scleroglucan of mean molecular weight 1·10⁶ to 12·10⁶. The weight ratio bactericide:glucan is usually from the range 1:10-10:1.

Native polysaccharides of the glucan class useful in the present invention usually are β-linked glucanes, such as 1,3-beta-glucanes e.g. of plant, bacterial or especially fungal origin. They often contain side chains in 6-position (1,3-1,6-β-glucan type), preferred glucanes of this type are scleroglucans known e.g. from U.S. Pat. No. 5,814,341, U.S. Pat. No. 6,162,447 and U.S. Pat. No. 6,162,449. The scleroglucan to be used in the present invention is to be understood as a product which may be isolated from the fungi Sclerotium, Lentillium or Schizophyllium (thus also called scleroglucan, lentinan, schizophyllan) and may be been obtained by cultivation of microorganisms in a manner as described in the US patents cited above, preferably microorganisms in the form of the plant-pathogenic fungi imperfecti Sclerotium rolfsii. In general, preferred features of the native polysaccharides of the glucan class useful in combination with the bactericide as described are identical with the scleroglucan classes described further above.

Compositions of the invention, especially those containing the lactate and/or 1,2-pentanediol component c), or glucan plus bactericide, are usually not prepared by adding the pure components a) and c) in the required amounts to the desired end formulation; instead, a concentrated formulation usually is prepared as a first step, containing component a) in a concentration that provides good handling especially with regard to the viscosity of the formulation, good storage stability, and easy dosability of the concentrate. The invention therefore also pertains to a concentrate for the preparation of a cosmetic or pharmaceutical formulation, which concentrate is characterized by containing

a) about 0.3 to 3% by weight, e.g. about 1.0% by weight, relative to the total weight of the concentrate, of the polysaccharide (scleroglucan as described above for component a),
b) a cosmetically or pharmaceutically acceptable carrier, which usually is an aqueous carrier such as water, physiological or near physiological solution of sodium chloride, or a suitable buffer solution, and
c) lactate and/or 1,2-pentanediol in a weight ratio relative to component (a) from the range 2:1 to 20:1;
and further to a concentrate, which may be characterized by containing
a) about 0.3 to 3% by weight, e.g. about 1.0% by weight, relative to the total weight of the concentrate, of the polysaccharide (e.g. scleroglucan as described above),
b) a bactericide as described above, and optionally
c) lactate and/or 1,2-pentanediol in the amount desired (see above), and
d) a cosmetically or pharmaceutically acceptable carrier, which usually is an aqueous carrier such as water, physiological or near physiological solution of sodium chloride, or a suitable buffer solution.

The concentrate may contain further components required for the preparation of the desired end formulation; often, however, it will consist essentially of these components as described above.

The cosmetic composition may constitute, e.g., a shampoo, rinse, gel and/or hair conditioner, hair-removal preparations (e.g. hair-removing powders, liquid hair-removing preparations, cream- or paste-form hair-removing preparations, hair-removing preparations in gel form or aerosol foams), cosmetic hair treatment preparations such as, e.g, hair-washing preparations in the form of shampoos and conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, hair foams, hairsprays, bleaching preparations, e.g. hydrogen peroxide solutions, lightening shampoos, bleaching creams, bleaching powders, bleaching pastes or oils, temporary, semi-permanent or permanent hair colourants, preparations containing self-oxidising dyes, or natural hair colourants, such as henna or chamomile, wherein the scleroglucan component a) optionally in combination with component c) may perform one or more of the following functions:

i) effect an improvement in the combability of hair treated with the shampoo/conditioner;
ii) effect an improvement in the dispersion of other components in the shampoo/conditioner; iii) act as a smoothing agent for hair treated with the shampoo/conditioner; and
iv) effect an improvement in the level of fixing of such additives as dyes or UV absorbers in the shampoo/conditioner.

The cosmetic or pharmaceutic composition according to the present invention may also constitute a skin care composition, e.g., anti-wrinkle-product; lip-care formulation; moisturizing cream; wound-care-formulation; skin-washing and cleansing preparations in the form of tablet-form or liquid soaps, soapless detergents or washing pastes; bath preparations, e.g. liquid (foam baths, milks, shower preparations) or solid bath preparations, e.g. bath cubes and bath salts; skin-care preparations, e.g. skin emulsions, multi-emulsions or skin oils; cosmetic personal care preparations, e.g. facial make-up in the form of day creams or powder creams, face powder (loose or pressed), rouge or cream make-up, eye-care preparations, e.g. eyeshadow preparations, mascara, eyeliner, eye creams or eye-fix creams; lip-care preparations, e.g. lipsticks, lip gloss, lip contour pencils, nail-care preparations, such as nail varnish, nail varnish removers, nail hardeners or cuticle removers; foot-care preparations, e.g. foot baths, foot powders, foot creams or foot balsams, special deodorants and antiperspirants or callus-removing preparations; light-protective preparations, such as sun milks, lotions, creams or oils, sunblocks or tropicals, pre-tanning preparations or after-sun preparations; skin-tanning preparations, e.g. self-tanning creams; depigmenting preparations, e.g. preparations for bleaching the skin or skin-lightening preparations; insect-repellents, e.g. insect-repellent oils, lotions, sprays or sticks; deodorants, such as deodorant sprays, pump-action sprays, deodorant gels, sticks or roll-ons; antiperspirants, e.g. antiperspirant sticks, creams or roll-ons; preparations for cleansing and caring for blemished skin, e.g. synthetic detergents (solid or liquid), peeling or scrub preparations or peeling masks; shaving preparations, e.g. shaving soap, foaming shaving creams, non-foaming shaving creams, foams and gels, preshave preparations for dry shaving, aftershaves or aftershave lotions; fragrance preparations, e.g. fragrances (eau de Cologne, eau de toilette, eau de parfum, parfum de toilette, perfume), perfume oils or perfume creams such as an emulsion or cream in which the scleroglucan a) optionally in combination with component c) may perform one or more of the following functions:

i) effect a lubricating function, thereby facilitating the spreading of the composition on the skin;
ii) act as a film-forming agent, thereby providing a protective film on the skin, which film, while almost undetectable by touching, provides the skin with a silky feel;
iii) effect a smoothing of the skin by reducing the scaling of the outermost layer of stratum corneum;
iv) effect an anti-inflammatory effect on the skin;
v) effect an improvement in the dispersion of other components of the skin care composition; and
vi) act as an emulsifier or co-emulsifier for the skin care composition.

The present antibacterial compositions are suitable for contact with the mucosa or tissues of the oral cavity; they may be formulated inter alia as an oral care or feminine hygiene composition such as a feminine hygiene washing lotion or spray, or a composition for the treatment of a medical, especially oral, implant (prior or after nidation), or a denture or brace. The present composition may also be used as an eye drop formulation, an eye make-up (e.g. eyeliner, eye cream or eye-fix cream) or an eye make-up remover.

Preferred are oral care compositions including tooth pastes or gels, mouth washes, gargles, inhalants, denture or implant or brace or cleaner thereof, adhesive paste.

In oral compositions, a combination with a further agent suitable to inhibit alkaline phosphatase is preferred. Such agents may, for example, be selected from polyanionic and polyanionically-derivatised “natural” polysaccharides, e.g. phosphochitosanes or especially phosphonochitosanes, as described in WO 99/32073.

The glucan often contains a further component (c) such as lactate and/or 1,2-pentanediol. The lactate often is sodium lactate. One part by weight of component c) is preferably used on 4 to 100, especially 5 to 20 parts by weight of the scleroglucan.

Specifically, amounts of component (c) relative to component (a) are often 1 to 15% by weight, especially 4 to 11% by weight of a lactate such as sodium lactate, and/or 4 to 20% by weight, especially 8 to 18% by weight, of 1,2-pentanediol, each relative to the β-1,3-scleroglucan (a).
Regarding end-product formulations, the skin care composition may be formulated as a wide variety of cosmetic or pharmaceutical preparations, for example: creams, gels, lotions, alcoholic and aqueous/alcoholic solutions, emulsions, wax/fat compositions, stick preparations such as lipsticks or deodorants, powders or ointments.
   in the form of liquid preparations as a W/O, O/W, O/W/O, W/O/W or PIT emulsion and all kinds of microemulsions,
      in the form of liquid crystalline structures represented either by hexagonal phase, by micellar cubic phase or by lamellar phase; among lamellar liquid crystals, there are oleosomes, hydrosomes and phosphosomes (structure built by the combination of surfactants and biomimetic phospholipids),
   in the form of a gel,
   in the form of an oil, a cream, milk or lotion,
   in the form of a powder, a lacquer, a tablet or make-up,
   in the form of a stick,
   in the form of a spray (spray with propellent gas or pump-action spray) or an aerosol,
   in the form of a foam, or
   in the form of a paste.

As water- and oil-containing emulsions (e.g. W/O, O/W, O/W/O and W/O/W emulsions or microemulsions) the preparations contain, for example, from 1 to 60% by weight, especially from 5 to 50% by weight and preferably from 10 to 35% by weight, based on the total weight of the composition, of at least one oil component, from 0 to 30% by weight, especially from 1 to 30% by weight und preferably from 4 to 20% by weight, based on the total weight of the composition, of at least one emulsifier, from 10 to 95% by weight, based on the total weight of the composition, of water, and from 0 to 88.9% by weight, especially from 1 to 50% by weight, of further cosmetically acceptable adjuvants.

The composition according to the present invention may also contain one or one more additional compounds as described below.

### Fatty Alcohols

Guerbet alcohols based on fatty alcohols having from 6 to 18, preferably from 8 to 10 carbon atoms including cetyl alcohol, stearyl alcohol, cetearyl alcohol, oleyl alcohol, octyldodecanol, benzoate of C12-C15 alcohols, acetylated lanolin alcohol, etc.

### Esters of Fatty Acids

Esters of linear C₆-C₂₄ fatty acids with linear C₃-C₂₄ alcohols, esters of branched C₆-C₁₃-carboxylic acids with linear C₆-C₂₄ fatty alcohols, esters of linear C₆-C₂₄ fatty acids with branched alcohols, especially 2-ethylhexanol, esters of hydroxycarboxylic acids with linear or branched C₆-C₂₂ fatty alcohols, especially dioctyl malates, esters of linear and/or branched fatty acids with polyhydric alcohols (for example propylene glycol, dimer diol or trimer triol) and/or Guerbet alcohols, for example caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeo-stearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerisation of unsaturated fatty acids) with alcohols, for example, isopropyl alcohol, caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoyl alcohol, linolenyl alcohol, elaeostearyl alcohol, arachidyl alcohol, gadoleyl alcohol, behenyl alcohol, erucyl alcohol and brassidyl alcohol and technical-grade mixtures thereof (obtained, for example, in the high-pressure hydrogenation of technical-grade methyl esters based on fats and oils or aldehydes from Roelen's oxosynthesis and as monomer fractions in the dimerisation of unsaturated fatty alcohols).

Examples of such ester oils are isopropylmyristate, isopropylpalmitate, isopropylstearate, isopropyl isostearate, isopropyloleate, n-butylstearate, n-hexyllaurate, n-decyloleate, isooctylstearate, iso-nonylstearate, isononyl isononanoate, 2-ethylhexylpalmitate, 2-hexyllaurate, 2-hexyldecylstearate, 2-octyldodecylpalmitate, oleyloleate, oleylerucate, erucyloleate, erucylerucate, cetearyl octanoate, cetyl palmitate, cetyl stearate, cetyl oleate, cetyl behenate, cetyl acetate, myristyl myristate, myristyl behenate, myristyl oleate, myristyl stearate, myristyl palmitate, myristyl lactate, propylene glycol dicaprylate/caprate, stearyl heptanoate, diisostearyl malate, octyl hydroxystearate, etc.

### Natural or Synthetic Triglycerides Including Glyceryl Esters and Derivatives

Di- or tri-glycerides, based on C₆-C₁₈ fatty acids, modified by reaction with other alcohols (caprylic/capric triglyceride, wheat germ glycerides, etc.). Fatty acid esters of polyglycerin (polyglyceryl-n such as polyglyceryl-4 caprate, polyglyceryl-2 isostearate, etc. or castor oil, hydrogenated vegetable oil, sweet almond oil, wheat germ oil, sesame oil, hydrogenated cottonseed oil, coconut oil, avocado oil, corn oil, hydrogenated castor oil, shea butter, cocoa butter, soybean oil, mink oil, sunflower oil, safflower oil, macadamia nut oil, olive oil, hydrogenated tallow, apricot kernel oil, hazelnut oil, borago oil, etc.

Waxes including esters of long-chain acids and alcohols as well as compounds having wax-like properties, e.g., carnauba wax, beeswax (white or yellow), lanolin wax, candellila wax, ozokerite, japan wax, paraffin wax, microcrystalline wax, ceresin, cetearyl esters wax, synthetic beeswax etc., or hydrophilic waxes such as Cetearyl Alcohol or partial glycerides.

### Hydrocarbon Oils:

Mineral oil (light or heavy), petrolatum (yellow or white), microcrystalline wax, paraffinic and isoparaffinic compounds, hydrogenated isoparaffinic molecules as polydecenes and polybutene, hydrogenated polyisobutene, squalane, isohexadecane, isododecane and others from plant or animal origin.

Further Components include

Silicones or siloxanes (organosubstituted polysiloxanes), including siloxanes (e.g. cyclic or polymeric), Silanol compounds or dimethiconols, Silicone elastomers & resins, Alkyl-Modified Siloxanes (AMS),

Fluorinated or perfluorinated oils,
Super-fatting agents,
Pearlescent waxes,
Anti-wrinkle actives, including sulfur-containing D and L amino acids, vitamin B compounds etc.,
Skin lightening agents
Deodorising active ingredients, for example, antiperspirants, Esterase inhibitors, Antibacterial active ingredients including chitosan, phenoxyethanol, chlorhexidine gluconate, 5-chloro-2-(2,4-dichlorophenoxy)-phenol (Triclosan®),
Consistency regulators/thickeners—Rheology modifiers, such as Natural thickeners, Mineral thickeners, Synthetic Rheology modifiers, Phospholipid derivatives;
Polymers, e.g. cationic polymers such as cationic cellulose derivatives, anionic, zwitterionic, amphoteric and non-ionic polymers;
Hydrotropic agents,
Perfume oils,
Emulsifiers, such as O/W emulsifiers, W/O emulsifiers, Non ionic emulsifiers such as PEG modified components, Anionic emulsifiers, Silicone emulsifiers (particularly suitable for W/Si emulsions);
see corresponding components published on Oct. 25, 2005 on ip.com under the identifier IPCOM000130489 D for further details.

The emulsifiers are often used in an amount of, for example, from 1 to 30% by weight, especially from 4 to 20% by weight and preferably from 5 to 10% by weight, based on the total weight of the composition.

When formulated in O/W emulsions, the preferably amount of such emulsifier system could represent 5% to 20% of the oil phase.

### Further Components Include:

### Adjuvants and Additives

alpha glucosylrutin (CAS No. 130603-71-3), 2-butyloctyl o-hydroxybenzoate (CAS No. 190085-41-7), vitamin E (CAS No. 1406-18-4), vitamin E acetate (CAS No. 58-95-7), diethylhexyl 2,6-naphthalate, di-n-butyl adipate, di(2-ethylhexyl)-adipate, di(2-ethylhexyl)-succinate and diisotridecyl acelaat, and also diol esters, such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate. Esters of C₆-C₂₄ fatty alcohols and/or Guerbet alcohols with aromatic carboxylic acids, saturated and/or unsaturated, especially benzoic acid, esters of C₂-C₁₂dicarboxylic acids with linear or branched alcohols having from 1 to 22 carbon atoms or polyols having from 2 to 10 carbon atoms and from 2 to 6 hydroxy groups, or iminodisuccinic acid and imiondisuccinic acid salts [CAS 7408-20-0] or latex particles, aloe vera, chamomile, ginko biloba, ginseng, coenzyme Q10, laminaria ochroleuca extract, magnolia oborata extract, melalenca alternifolia leaf oil, rubus idaeus seed oil, vaccinium macrocarpon seed oil, pumpkin seed extract, pumpkin seed oil, grape seed extract, carnosine, alpha-arbutin, madecassoside, termino-laside, tetrahydrocurcuminoids (THC), mycosporines, mycosporine like amino acids from the red alga porphyra umbilicalis, mycosporine-like amino acids (as described in WO2002039974), cis-9-octadecenedioic acid, lipoic acid, laurimino dipropiomic acid tocopheryl phosphates (LDTP), microcrystalline cellulose (MCC), polycarbonates as described in WO 0341676, sterols (cholesterol, lanosterol, phytosterols), as described in WO0341675 and linear poly-alpha-glucans as described in U.S. Pat. No. 6,616,935

It is furthermore possible for the cosmetic preparations to contain, as adjuvants, anti-foams, such as silicones, structurants, such as maleic acid, solubilisers, such as ethylene glycol, propylene glycol, glycerol or diethylene glycol, opacifiers, such as latex, styrene/PVP or styrene/acrylamide copolymers, complexing agents, such as EDTA, NTA, alaninediacetic acid or phosphonic acids, propellants, such as propane/butane mixtures, N₂O, dimethyl ether, CO₂, N₂ or air, so-called coupler and developer components as oxidation dye precursors, reducing agents, such as thioglycolic acid and derivatives thereof, thiolactic acid, cysteamine, thiomalic acid or mercaptoethanesulfonic acid, or oxidising agents, such as hydrogen peroxide, potassium bromate or sodium bromate.

### Hydrating Agents

glycerol, sorbitol, lactic acid, alpha-hydroxiacids, hyaluronic acid, chitosan, glycosaminoglycans and its breakdown products and sugars especially C6 and C5 sugars such as glucose, lactose, trehalose, and arabinose, desoxyribose, xylose, pyrrolidone carboxylate, oligopeptides and aminoacids containing or being preferentially Alanine, Asparagine, beta-Alanine, Citrulline Glutamic acid, Histidine Leucine Lysine, Ornithine, Phenylalanine Serine, Threonine, Valine,

### Antioxidants

The topical application could contain at least one hydrophilic or lipophilic antioxidant within the concentration range from 0.001% to 10% of the total weight of the cosmetic preparation.

Those antioxidants are preferably selected from the group containing:
tocopherol (α, γ, γ, δ isomers) and its esters of acids with general formulas

H(CH₂)n(CHR)COOH (1)

CH₃(CH₂)mCH═CH(CH₂)nCOOH (2)
where R is hydrogen atom or OH group, m, n are integral numbers from 0 to 22 where m+n sum is maximally 22.
tocotrienol (α, β, γ, δ isomers), containing one unsaturated fatty chain, and its esters of acids
ascorbic acid and its esters of acids such as phosphoric acid and also sodium, potassium, lithium and magnesium salts, Ascorbyl Tetraisopalmitate, further ester with pyrrolidoncarboxylic acid and esters of acids with general formulas

H(CH₂)n(CHR)COOH (3)

CH₃(CH₂)mCH═CH(CH₂)nCOOH (4)
where R is hydrogen atom or OH group, m, n are integral numbers from 0 to 20 where m+n sum is maximally 21.
Retinoids include all natural and/or synthetic analogs of vitamin A or retinal-like compounds which possess the biological activity of vitamin A in the skin as well as the geometric isomers and stereoisomers of these compounds. Preferred compounds are retinal, retinol esters (e.g., C2-C22 alkyl esters (saturated or unsaturated alkyl chains) of retinal, including retinyl palmitate, retinyl acetate, retinyl propionate), retinal, and/or retinoic acid (including all trans retinoic acid and/or 13-cis-retinoic acid) or derivatives. Other retinoids which are useful herein are described in U.S. Pat. No. 4,677,120, issued Jun. 30, 1987 to Parish et al; U.S. Pat. No. 4,885,311, issued Dec. 5, 1989 to Parish et al; U.S. Pat. No. 5,049,584, issued Sep. 17, 1991 to Purcell et al., U.S. Pat. No. 5,124,356, issued Jun. 23, 1992 to Purcell et al. Other suitable retinoids are tocopheryl-retinoate [tocopherol ester of retinoic acid (trans or cis)], adapalene [6-(3-(1-adamantyl)-4-methoxyphenyl)-2-naphtoic acid] and tazarotene (ethyl 6-[2-(4,4-dimethylthiochroman-6-yl)-ethynyl]nicotinate);
carotenoids such as α-, β-, γ-, and δ-carotene, lutein, xanthophylls, zeaxanthine, astaxanthin, violaxanthine, cryptoxanthine, fukoxanthine, antheraxanthine, lycopene, didehydrolycopene and tetradehydrolycopene carotenoids
enzymatic antioxidants such as Glutathione peroxidase, Catalase, Superoxide dismutase.
Ubiquinone and Idebenone (hydroxydecyl Ubiquinone), Ubiquinol and its derivatives
Lipoic acid and its derivatives such as alpha-lipoic acid . . . .
Rutinic acid and its derivatives such as α-glucosylrutin, a water soluble flavonoid, rutin hydrate (vitamin P)
Botanical extracts such as white and green tea extracts, chicory leaf extract (Cichorium intubybus), Passionflower extract (Passiflora incarnata), Aspalathus linearis extract, rosmary extract, red leaf extract of Aceraceae Maple tree or of Rosaceae Chemy tree, Curcuma longa L (curcuminoids active ingredients), Leontopodium alpinum extract, Emblica officinalis (phyllanthus emblica) tree extract . . . .
Phenolic acids such as caffeic acid, 3,4-dihydroxyphenyl acetic acid, 3,4-dihydroxybenzoic acid.
Flavonoids and polyphenols such as flavanones selected from the group consisting of unsubstituted flavanones, mono-substituted flavanones, and mixtures thereof; chalcones selected from the group consisting of unsubstituted Chalcones, non-substituted chalcones, di-substituted chalcones, tri-substituted chalcones, and mixture thereof; flavones selected from the group consisting of unsubstituted flavones, mono-substituted flavones, di-substituted flavones, and mixtures thereof; one or more isoflavones; coumarins selected from the group consisting of unsubstituted coumarins, mono-substituted coumarins, di-substituted coumarins, and mixtures thereof; flavonols, anthocyanins, catechins, proanthocyanidins (Grape seed extract). Flavonoids which are broadly disclosed in U.S. Pat. Nos. 5,686,082 and 5,686,367 can also be used.
chlorogenic acid and ferulic acid.

It is also possible to use a second kind of antioxidants that interrupt the photochemical reaction chain triggered when UV radiation penetrates the skin or hair. Typical examples of such antioxidants are amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), aurothioglycose, propylthiouracil and other thiols (e.g. thioredoxin, glutathione, cysteine, cystine, cystamine and the glycosyl, N-acetyl, methyl, ethyl, propyl, amyl, butyl, lauryl, palmitoyl, oleyl, linoleyl, cholesteryl and glyceryl esters thereof) and also salts thereof, dilauryl thiodipropionate, distearyl thiodipropionate, thiodipropionic acid and derivatives thereof (esters, ethers, peptides, lipids, nucleotides, nucleosides and salts) and also sulfoximine compounds (e.g. buthionine sulfoximines, homocysteine sulfoximine, buthionine sulfones, penta-, hexa-, hepta-thionine sulfoximine).

But also (metal) chelating agents (e.g. hydroxy fatty acids, palmitic acid phytic acid, lactoferrin) and preferably those disclosed in U.S. Pat. No. 5,487,884, issued Jan. 30, 1996 to Bisset et al; International publications No. 91/16035 & No. 91/16034 from Bush et al., published Oct. 31, 1995. Hydroxy acids (e.g. citric acid, lactic acid, malic acid), humic acid, bile acid, bile extracts, bilirubin, biliverdin, EDTA, EDDS, EGTA and derivatives thereof, unsaturated fatty acids and derivatives thereof (e.g. linolenic acid, linoleic acid, oleic acid), folic acid and derivatives thereof, coniferyl benzoate of benzoin resin, ferulic acid, furfurylidene glucitol, carnosine, butyl hydroxytoluene, butyl hydroxyanisole, nordihydroguaiaretic acid, trihydroxy-butyrophenone, uric acid and derivatives thereof, mannose and derivatives thereof, N-[3-(3,5-di-tert-butyl-4-hydroxyphenyl)propionyl]sulfanilic acid (and salts thereof, for example the disodium salts), zinc and derivatives thereof (e.g. ZnO, ZnSO₄), selenium and derivatives thereof (e.g. selenium methionine), stilbene and derivatives thereof (e.g. stilbene oxide, trans-stilbene oxide) and the derivatives suitable according to the invention (salts, esters, ethers, sugars, nucleotides, nucleosides, peptides and lipids) of those mentioned active ingredients. HALS (=“Hindered Amine Light Stabilizers”) compounds may also be mentioned; components of those latest categories, carbon or ester/amide bridged phenols or lactones thereof, or some sterically hindered amines as disclosed in PCT patent application No. EP2005/055475 of Oct. 24, 2005, or published on Oct. 25, 2005 on ip.com under the identifier IPCOM000130489D.

### Anti-Inflammatory Agents

The topical application could additionally contain at least one component with anti-inflammatory effect, preferably from 0.1% to 10% more preferably about 0.5% to about 5%, of the composition, from following groups:
Steroidal anti-inflammatory agents, including but not limited to, corticosteroids such as hydrocortisone and their derivatives . . . .
Non-steroidal anti-inflammatory agents, including but not limited to, oxicams, salycilates, acetic acid derivatives, fenamates, propionic acid derivatives, pyrazoles . . .
Natural anti-inflammatory agents including but not limited to:
   α-bisabolol, allantoin, lyophilized extract of aloe vera, panthenol, betulin, compounds of the Licorice (Glycyrrhiza glabra) including glycyrrhetic acid, glycyrrhizic acid, and derivatives thereof (salts and esters) such as sodium glycyrrhizinate, potassium glycyrrhizinate, ammonium glycyrrhizinate
   botulinic acid, alkaline salts thereof and salts of alkaline-earth metals, boswellic acid, alkaline salts thereof and salts of alkaline-earth metals, rosemaric acid, alkaline salts thereof and salts of alkaline-earth metals
   polynonsatured fatty acids, as linoleic (18:2n6), α-linolenic (18:3n3), γ-linolenic (18:3n6), octadekanetetraenic (18:4n3), dihomo-γ-linolenic (20:3n6), eikosantetraenic (20:4n3), arachidonic (20:4n6), eikosanpentaenic (20:5n3) acids and esters thereof with alcohols of the general formula

R1(CH2)m-(CHOH)—(CH2)nR2 (5)
where R1 and R2 are hydrogen atoms or OH group, m, n are integral numbers from 0 to 17 where m+n sum is maximally 21;
phytosterols and their polyethoxylate derivatives of the general formulas (6) and (7) below, where R is isoalkyl or isoalkenyl group with 8-10 carbon atoms, where n is integral number from 0 to 50, especially campesterol, β3-sitosterol, stigmasterol, cholesterol, Δ-5-avenasterol, Δ-7-avenasterol, brassicasterol, spinasterol and fukosterol

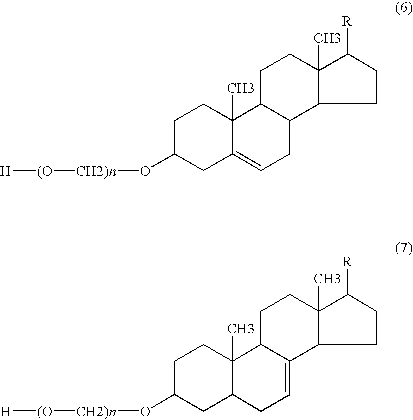

### Preservatives and Bacteria-Inhibiting Agents

Suitable preservatives include, for example, Methyl-, Ethyl-, Propyl-, Butyl-parabens, Benzalkonium chloride, 2-Bromo-2-nitro-propane-1,3-diol, Dehydroacetic acid, Diazolidinyl Urea, 2-Dichloro-benzyl alcohol, DMDM hydantoin, Formaldehyde solution, Methyldibromoglutanitrile, Phenoxyethanol, Sodium Hydroxymethylglycinate, Imidazolidinyl Urea, Triclosan, Silver Dihydrogen Citrate and further substance classes listed in the following reference: K. F. DePolo—A short textbook of cosmetology, Chapter 7, Table 7-2, 7-3, 7-4 and 7-5, p 210-219.

Typical examples of bacteria-inhibiting agents are preservatives that have a specific action against gram-positive bacteria, such as 2,4,4′-trichloro-2′-hydroxydiphenyl ether, chlorhexidine (1,6-di(4-chlorophenyl-biguanido)hexane) or TCC (3,4,4′-trichlorocarbanilide). Silver Dihydrogen Citrate is also exhibiting good bacteria-inhibiting property. A large number of aromatic substances and ethereal oils also have antimicrobial properties. Typical examples are the active ingredients eugenol, menthol and thymol in clove oil, mint oil and thyme oil. A natural deodorising agent of interest is the terpene alcohol farnesol (3,7,11-tri-methyl-2,6,10-dodecatrien-1-ol), which is present in lime blossom oil. Glycerol monolaurate has also proved to be a bacteriostatic agent. The amount of the additional bacteria-inhibiting agents present is usually from 0.1 to 2% by weight, based on the solids content of the preparations.

### Colourants

There may be used as colourants the substances that are suitable and permitted for cosmetic purposes, as compiled, for example, in the publication “Kosmetische Färbemittel” of the Farbstoffkommission der Deutschen Forschungsgemeinschaft, Verlag Chemie, Weinheim, 1984, pages 81 to 106. The colourants are usually used in concentrations of from 0.001 to 0.1% by weight, based on the total mixture.

### Anti-Dandruff Agents

As anti-dandruff agents there may be used, for example, climbazole, octopirox and zinc pyrithione. Customary film formers include, for example, chitosan, microcrystalline chitosan, quaternised chitosan, polyvinylpyrrolidone, vinylpyrrolidone/vinyl acetate copolymers, polymers of quaternary cellulose derivatives containing a high proportion of acrylic acid, collagen, hyaluronic acid and salts thereof and similar compounds.

The active ingredients of present components a) and c) or even other actives such as antioxidants, anti-inflammatory agents, anti-wrinkle ingredients, skin lightening agents, etc. may be integrated in a cosmetically or dermatologically acceptable carrier system within the cosmetic end-product (cosmetic preparation). Several particulate skin care delivery systems are proposed:

A] Nanoemulsions and nanoparticles are mainly based on lecithin or fractionated phospholipids;
nanoemulsions with particle size range of 20 nm-50 nm represented by a single layer membrane:
Nanotopes are lipid core surrounded by a membrane composed of phospholipids and co-surfactants; stable and small size particle (smaller than liposomes) due to the intecalation of co-surfactant between the extending lecithin molecules
Nanosomes
nanoparticles with particle size range of 100 nm-300 nm represented by a bi-layer membrane:
Liposomes are spherical vesicles which consist of amphiphilic lipids (predominantly phospholipids) enclosing on aqueous core and forming one or several concentric bi-layers; small unilamellar vesicles (SUV), large unilamellar vesicles (LUV), large multilamellar vesicles (MLV) or multivesicullar vesicles (MVV). The incorporation of the antioxidant composition could be achieved into the bi-layers, into the aqueous core or distributed in both.
   B] Microcapsules with particle size >1 μm based on matrix or encapsulation layer;

Spherical system based on a core material containing the active. The core is, then surrounded by one or several coating layers or shells.

Polymers used to form those microcapsules include natural gums, cellulosic ingredients, polysaccharides, synthetic polyacrylates or polyacrylamides or even polyvinyl alcohol (PVA), but also lipids, inorganics (silicates/clays) and high molecular weight proteins such as gelatin, albumin . . .
Nylon micro-porous spheres (Orgasol range from Elf Atochem)
Mineral fillers such as sericite surface-treated by bifunctional coating; reaction between reactive fatty acid derivatives and the aqueous solution of sericite.
Glycospheres; core based on modified starch and outer lipid membrane based on fatty acids and polar lipids
Silica shells, made of silicates, for non aqueous and solid end-products such as sticks, dry powders . . . .
   C] Matrix particulate systems which entrap the active ingredient within the uniform core matrix;
Solid Lipid Nanoparticle (SLN) technology based only on solid lipids
Nanostructured Lipid Carriers (NLC) made of blend of solid lipid and liquid lipid [symbol] particle size diameter within the range 80 nm-1 μm
Nanospheres (patent Nanosal U.S. Pat. No. 6,491,902) represented by solid hydrophobic and highly cationic nanospheres (particle size range 10 nm to 1 μm) and based on solid hydrophobic matrix coated with highly cationic or bioadhesive layer
   D] Multi-walled delivery systems

They are similar to liposome structures but made only of non-phospholipidic “membrane-mimetic” amphiphiles such as oleic acid, saturated or unsaturated fatty acids, long-chain soaps combined with non ionic surfactants, derivatives of polyglycerol, di-ammonium amphiphiles, cationic surfactants, cationic amphiphiles involving aminoacid residues, sucrose fatty acid esters, aqueous mixture of anionic and cationic surfactants.

E] Microsponge technology

Such system is based on microscopic polymer-based sphere that consist of myriad of interconnecting voids within a non collapsible structure (non continuous shell); example are copolymer of styrene and divinylbenzene, or vinyl derivatives, water-swellable particles made of lactose, cellulose and cellulose derivatives such as Unispheres from Induchem . . . .

F] Silicone-based vesicles

These are multi-layers vesicles similar as liposome structures where layers are made of polyether-modified dimethicone (dimethicone copolyol), silicone elastomers or blends of dimethicone crosspolymer, dimethicone/vinyldimethicone crosspolymer or PEG-modified dimethicone crosspolymer

### G] Cyclodextrins

Oligomeric and cyclic carbohydrate compounds containing 6 to 8 glucose units; α-, β- and γ-cyclodextrin

The composition according to the present invention may also constitute detergent compositions such as any shampoo, shower-gel, foaming bath, body wash preparations with cleansing, washing, conditioning or scrubbing effects on skin and/or hair.

The cosmetic detergent composition should contain water deionised or a mix of water and acceptable cosmetic solvent such as C1-C4 alcohols (ethanol, isopropanol, tertiobutenol, n-butanol) or glycols such as propylene glycol, glycol ethers . . . .

The water content should be between 40% and 95% of the total weight of the final composition. The total amount of surfactants (detergent agents) should represent from 4% to 50%, and preferably from 6% to 35% of the total weight of the final composition.

The cosmetic detergent composition should have a pH from 3 to 10, and preferably adjusted between 4 and 8 with basic solutions such as Monoethanolamine, Diethanolamine, Triethanolamine, isopropanolamine or propanediamine-1,3, but also with acidic solutions such as citric acid, lactic acid, sorbic acid, phosphoric acid or glycolic acid.

Usually cosmetic detergent products contain the following ingredients:
surfactants
thickeners and polymers as previously described for emulsion end-products
foam stabilizers and boosters
diluents such as water or polyols or other hydrotopic agents as previously described for emulsion end-products
additives such as super fatting agents, anti-wrinkle or skin lightening agents, biogenic active ingredients as previously described for emulsion end-products
pearlizers/opacifiers
colorants as previously described for emulsion end-products
perfumes as previously described for emulsion end-products
preservatives and bacteria-inhibiting agents as previously described for emulsion end-products
complexing agents (EDTA, NTA) and reducing agents

Surfactants, such as Anionic surfactants, Non ionic surfactants, Amphoteric or Zwitterionic surfactants, Cationic surfactants, are known components, mainly as published in on Oct. 25, 2005 on ip.com under the identifier IPCOM000130489D (see there for further details).

### FORMULATION EXAMPLES

In the following examples, the aqueous solution of Scleroglucan used contains the scleroglucan of Sclerotium rolfsii, and is one of the following concentrates:
a 1.0% b.w. aqueous solution of said scleroglucan (formulation A) or
formulation A mixed with sodium lactate to finally contain 7.5% b.w. of sodium lactate (formulation B) or
formulation A mixed with 1,2-pentanediol to finally contain 12.5% b.w. of 1,2-pentanediol (formulation C) or
formulation A, to which sodium lactate and 1,2-pentanediol is added, the final aqueous solution containing 7.5% by weight of sodium lactate and 12.5% by weight of 1,2-pentanediol (formulation D).

### Conditioning Shampoo

The requirements of a shampoo are to clean hair and scalp of soils and dirt; ingredients must be safe (low toxicology, low sensitization and low skin/eye irritation potential) and the detergents must present low substantivity.

The main ingredients in this application fields are:

### Primary Surfactants

Alkyl Sulfates, such as Ammonium Lauryl Sulfate, TEA Lauryl Sulfate, Sodium Lauryl Sulfate Ammonium Laureth Sulfate, Sodium Laureth Sulfate, Sodium C14-16 Olefin Sulfonate;

Sulfosuccinates, such as Disodium monlaurethsulfosuccinate, Disodium monolauramido MEA sulfosuccinate, Disodium monoleamido PEG-2 sulfosuccinate;
N-Acyl Sarcosinates, such as Sodium Cocoyl Sarcosinate, Sodium Lauroyl Sarcosinate;
N-Acyl Methyltaurates, such as Sodium N-methyl Cocoyl Taurate;
Amphoterics, such as Sodium Cocoamphoacetate, Sodium Cocoamphodiacetate, Sodium Cocoamphopropionate.
Secondary surfactants

Betaines, such as Cocamidopropyl betaine, Oleamidopropyl betaine, Isostearamidopropyl betaine;

Amides, such as Lauramide DEA, Cocamide DEA;

Foam Stabilizers, such as Lauramide DEA, Cocamide DEA, Cocamine Oxide; pH adjusting agents, such as Citric, Lactic, Sorbic, Phosphoric or Glycolic acids.

| Ingredients | |
|---|---|
| Primary surfactants (listed | 5%-10% |
| previously) | |
| Secondary surfactants (listed | 5%-15% |
| previously) | |
| Foam Stabilizers (listed previously) | 0%-5% |
| Water deionized | 40%-70% |
| Actives | 0%-10% |
| Conditioners | |
| Refatting agents | |
| Moisturizing agents | |
| Thickeners/Rheology mofifiers | 0%-3% |
| Humectants | 0%-2% |
| Aqueous Scleroglucan formulation | 0.1%-5% |
| PH adjusting agents | 0%-1% |
| Preservatives | 0.05%-1% |
| Perfume oils | 0.1%-1% |
| Antioxidants | 0.05%-0.20% |
| Chelating Agents (EDTA) | 0%-0.2% |
| Opascifying agents | 0%-2% |

### Shower-Gel

| Ingredients | |
|---|---|
| Sodium Laureth Sulfate | 15%-18% |
| Lauryl Glucoside | 4%-6% |
| Decyl Glucoside | 4%-6% |
| Cocamidopropyl Betaine | 1%-5% |
| Sodium Cocoamphoacetate | 0%-2% |
| PEG-200 Glyceryl Palmate | 0%-2% |
| PEG-150 | 4%-6% |
| PEG-240 | 8%-10% |
| PEG-220 | 5%-6% |
| Triethyl Citrate | 2%-9% |
| Glyceryl Mono Oleate | 1%-2% |
| Decyl Oleate | 0%-2% |
| Polyquaternium-7 | 0.05%-0.20% |
| Potassium Citrate | 4%-12% |
| Iminodisuccinate | 3%-5% |
| PEG-40 Hydrogenated Castor Oil | 0%-2% |
| Polyether-1 | 1%-4% |
| Aqueous Scleroglucan formulation | 0.1%-5% |
| Perfume | q.s |
| Preservative | q.s |
| Water deionized | Qsp 100 |

### Pearlized Mild Body Wash

| Ingredients | |
|---|---|
| Sodium Laureth Sulfate | 15% |
| Cocoyl Isethionate | 4%-6% |
| Sodium Lauroamphoacetate | 4%-6% |
| Sodium Xylene Sulfonate | 1%-5% |
| Sodium Methyl Cocoyl Taurate | 1%-5% |
| Acrylates Crosspolymer | 1.5% |
| Water deionized | 14.5% |
| Dimethiconol and TEA- | 4% |
| Dodecylbenzenesulfonate | |
| Sodium Cocoamphoacetate | 2% |
| Polyquaternium-7 | 2% |
| Water deionized | 5% |
| Sodium Hydroxide (18% solution) | 0.05% |
| Guar Hydroxypropyl trimonium | 0.15% |
| Chloride | |
| Citric acid (50% solution) | 0.05 |
| Water deionized | Qsp 100 |
| Mica and Titanium Dioxide | 0.2% |
| Aqueous Scleroglucan formulation | 0.1%-5% |
| Perfume | 0.5% |
| Citric acid (50% solution) | 0.6% |
| Preservative | 0.5% |

### Clear Shampoo

| Ingredients | |
|---|---|
| Sodium Lauryl Sulfate | 1% |
| Sodium Polyoxyethylene lauryl | 12% |
| sulfate | |
| Lauroamidopropyl betaine | 1% |
| POE(16) lauryl ether | 1% |
| Glyceryl monoisodecyl ether | 0.5% |
| Cationized cellulose | 0.5% |
| Polyvinyl Alcohol | 0.2% |
| Glycerin | 10% |
| Tocopherol Acetate | 0.24% |
| Sodium Sulfate | 3% |
| Aqueous Scleroglucan formulation | 0.1%-5% |
| Perfume | 0.1% |
| Sodium Hydroxide | q.s pH = 7 |
| Water deionized | Qsp 100 |

Clear Bath Gel with Suspended Beads

| Ingredients | |
|---|---|
| Sodium Laureth Sulfate (28% | 30% |
| solution) | |
| Acrylates Crosspolymer (30% | 10% |
| solution) | |
| Propylene Glycol | 2% |
| Sodium Hydroxide (18% solution) | 1.9% |
| Disodium EDTA | 0.1% |
| Benzophenone-4 | 0.02% |
| Cocamidopropyl Betaine (35% | 4% |
| solution) | |
| Polysorbate 20 | 0.8% |
| White beads | 1% |
| Aqueous Scleroglucan formulation | 0.1%-5% |
| Perfume | 0.6% |
| Citric acid (50% solution) | 0.6% |
| Preservative | 1% |
| Water deionized | Qsp 100 |

### Opthalmological Preparation

When the β-1,3-scleroglucan is used in an opthalmological preparation, it may be used together with other components such as:

a) opthalmological active ingredients e.g. Gentamicin sulphate, Lomefloxacin hydrochloride, Chloramphenicol, Sodium Diclofenac, Potassium Diclofenac, Dexamethason di-sodium phosphate, Naphazolin nitrate, Tetryzolin hydrochloride, Antazolin hydrochloride, Antazolin sulphate, Pilocarpin chloride, Vitamin A-palmitate and zinc sulphate;
b) opthalmological buffers such as boric acid, borax, acetic acid, sodium acetate, phosphoric acid, sodium dihydrogen phosphate, disodium hydrogen phosphate, sodium phosphate, Trometamol, citric acid and sodium citrate;
c) opthalmological preservatives such as benzyl alkylammonium chloride, benzoxonium chloride, chlorhexidine digluconate, chlorobutanol, phenylethyl alcohol and Thiomersal;
d) solvents such as ethanol, glycerol, polyethylene glycol and water or mixtures thereof;
e) solution aids such as Cremophor EL, Cremophor RH, Tween 20 and Tween 80;
f) isotonising agents such as sodium chloride, mannitol and sorbitol,
g) chelate formers such as disodium EDTA;
h) antioxidants such as α-tocopherol acetate, ascorbic acid, N-acetyl-cystine, sodium bisulphite, sodium thiosulphate and propyl gallate; and
i) viscosity-increasing compounds such as methylhydroxypropyl cellulose, saccharose, Carbopol 934P, Carbopol 940, Carbopol 980 and Polaxomer F127.

An aqueous opthalmological preparation may be formulated e.g. from the following ingredients:
100 mg of a scleroglucan formulation A-D (see above formulation examples)
1 mg Sodium Diclofenac
50 mg Solution aid (Cremophor EL)
6 mg Opthalmological buffer (Trometamol)
19 mg Boric acid
0.04 mg Opthalmological preservative (Thiomersal)
Water for injection purposes to 1.00 ml.

### Oral Care Preparation

The composition according to the present invention may also constitute an oral care preparation, e.g. a dental gel, a denture fixation aid, mouth rinse, or a tooth paste; a mucosal lubricant formulation such as a vaginal cream or gel; or an opthalmological preparation e.g. selected from eye drops, artificial tear or saliva formulations etc., in which the glucan component a) optionally in combination with component c) may perform one or more of the following functions:

i) effect lubrication of dry mucosae;
ii) effect thickening of liquid preparations;
iii) effect retention of active ingredients by formation of films on mucosal surfaces; and
iv) effect an improvement in the dispersion of other components in the composition.

The composition according to the present invention may also be used as e.g. a dental gel, a denture fixation aid, mouth rinse, or a tooth paste.

Such compositions are dealing to treat the hard and soft tissues surfaces of the oral cavity. The oral care and hygiene contribute to maintain prophylactic, therapeutic and cosmetic benefits that include reduction in caries, in plaque, in gingivitis and in tartar; treating hypersensitivity; freshening breath; whitening teeth and/or removing stains; remineralising teeth and the like.

Several product forms are proposed such as dentifrices or toothpastes, mouthwashes, chewing gums containing for example teeth whitening actives, anti-caries actives, breath freshening. . . . All those actives can be encapsulated by an encapsulating material that protects them from the other ingredients and also from environment agressions; encapsulating materials were already described in previous chapters. Nevertheless, we could mention Cyclodextrin, gum arabic, gelatin, casein, albumin, fibrinogen, xanthan gum, haemoglobin, soluble collagen peptides, sodium alginate, carboxymethyl cellulose, carrageenan, plyvinylpyrrolidone and similar natural or synthetic polymeric materials. Typically, the encapsulated active will comprise from 0.01% to 10% of the whole oral composition. Oral care products can contain a variety of optional components suitable for rendering such composition more cosmetically acceptable:

A]—organic surface active agents such as detersive material which imparts to the composition detersive and foaming properties; e.g.
Anionic surface active agents such as water soluble salts of higher fatty acid monoglyceride monosulphates, water soluble salts of higher alkyl sulphates, water soluble salts of alkyl aryl sulphonates, olefin sulfonates (olefin group contains 12-21 carbon atoms), alkylsulphoacetates, higher fatty acid ester of 1,2-dihydroxy propane sulphonates, acylamino acid and salts . . . as described in previous chapters.
Nonionic surface active agents as described in previous chapters and more especially the esters category.
Cationic surface active agents as described in previous chapters and more especially the quaternized imidazole derivatives, the tetraalkyl ammonium salts.
   B]—thickeners and viscosity modifiers, such as
natural thickeners as described in previous chapters
block polymers of ethylene oxide and propylene oxide, and other polymers as described in previous chapters
silica and silica derivatives as described in previous chapters.
   C]—pH adjusting and buffering agents, such as

Citric acid, succinic acid, phosphoric acid, sodium hydroxide, sodium carbonate.

D]—Flavouring agents, such as

Oil of peppermint, sassafras, cassia, clove bud oil, menthol, anethole, thymol, methyl salicylate, eucalyptol, 1-menthyl acetate, sage, eugenol, parsely oil, oxanone, oil of wintergreen, alpha-irisone, oil of spearmint, marjoram, lemon, orange, propenyl guaethol, cinnamon, and mixtures thereof.

E]—Humectant material, such as

Glycerin, sorbitol, propylene glycol, xylitol, lactilol, but also ethyl alcohol, mineral oil, corn syrup, glucose and inert sugars, glycols, honey.

The compositions of the invention usually contain a cosmetically or pharmaceutically acceptable carrier and/or further components or actives known for the purpose; for example flavours, colorants, sweeteners etc.; components useful for oral care compositions are, for example, as mentioned in JP-A-2001031541 and WO 99/32073.

### Examples for Oral Care Actives are:

Teeth colour modifying substances;
particles that when applied on the teeth surface modify the surface in terms of absorption and/or reflection of light. These include pigments colourants such as inorganic white pigments, inorganic coloured pigments, pearling agents, filler powders and the like (JP Kokai patent application No. 9 (1997)-100215, publish. Apr. 15, 1997); specific examples are selected from talc, mica, magnesium carbonate, calcium carbonate, magnesium silicate, aluminium magnesium carbonate, silica, titanium dioxide, zinc oxide, red iron oxide, brown iron oxide, yellow iron oxide, black iron oxide, ferric ammonium ferrocyanide, manganese violet, ultramarine nylon powder, polyethylene powder, methacrylate powder, polystyrene powder, silk powder, crystalline cellulose, starch, titanated mica, iron oxide titanated mica, bismuth oxychloride, and mixtures thereof. The levels of those particles are generally used in the range of about 0.05% to about 20%, by weight, of the oral care composition.
materials that remove or bleach intrinsic or extrinsic stains on or in teeth surfaces. These include peroxides, metal chlorites, perborates, percarbonates, peroxyacids, persulphates, and combinations thereof. Suitable peroxide compounds include hydrogen peroxide, urea peroxide, calcium peroxide, carbamide peroxide and mixtures thereof. Suitable metal chlorites include calcium chlorite, barium chlorite, magnesium chlorite, lithium chlorite, sodium chlorite and potassium chlorite. The levels of those agents are generally used in the range of about 0.1% to 30%, by weight, of the oral care composition.
Anti tartar agents;
pyrophosphates which include pyrophosphate salts, dialkali metal pyrophosphate salts, tetra-alkali metal pyrophosphate salts and mixtures thereof. Disodium dihydrogen pyrophosphate (Na2H2P2O7), tetrasodium pyrophosphate (Na4P2O7) and tetrapotassium pyrophosphate (K4P2O7); detailed description in Kirk & Othermer, Encylopeadia of Chemical Technology, 3^{rd} Ed., vol. 17,1982.
Polyphosphates; detailed description in U.S. Pat. No. 4,590,066, Parran & Sakkab, May 20, 1986.
Polyacrylates and other polycarboxylates; detailed description in U.S. Pat. No. 3,429,963, Shedlovsky, Feb. 25, 1969 and U.S. Pat. No. 4,661,341, Benedict & Sunberg, Apr. 28, 1987.
Polyepoxysuccinates; detailed description in U.S. Pat. No. 4,846,650, Benedict & Sunberg, Jul. 11, 1989.
Polyphosphonates; detailed description in U.S. Pat. No. 4,877,603, Degenhardt & Kozikowski, Oct. 31, 1989.

Preferred anti tartar agents are the linear “glassy” polyphosphates having the formula:

XO(XPO₃)ₙX

wherein X is sodium, potassium, or hydrogen and n averages from about 6 to about 125.
Anti-plaque agents; substances that inhibit the accumulation of bacterial deposits on the surfaces of the oral cavity

Examples include xylitol and other anti-microbial agents as described in previous chapters.
Fluoride ion source; well known as anticaries agents, such as
Alkali metal monofluorophosphates that include sodium monofluorophosphate, lithium monofluorophosphate, potassium monofluorophosphate, ammonium monofluorophosphate.
Alkali metal monofluoropolyphosphates such as Na4P2O9F, K4P3O9F, (NH4)₄P3O9F, Na3 KP3O9F, (NH4)₃NaP3O9F and Li4P3O9F.

Preferred fluoride ion sources include sodium fluoride, potassium fluoride, stannous fluoride, ammonium fluoride and mixtures thereof. The levels of those agents are generally used in the range of about 50 ppm to 10 000 ppm within the oral care composition.
Anti-microbial agents; such as described in previous chapters;
especially for cosmetic/skin care applications, preferably, 5-chlor-2-(2,4-dichlorophenoxy)-phenol (Triclosan), Silver Dihydrogen Citrate, Phthalic acid and its salts, alexidine, hexetidine, sanguinarine, benzalkonium chloride, salicylamilide, domiphen bromide, cetylpyridinium chloride, tetradecylpyridinium chloride, N-tetradecyl-4-ethylpyridinium chloride, octenifine, delmopinol, octapinol and other piperidine derivatives, nicin preparations, zinc/stannous ion agents, essential oils including thymol, geraniol, carvacrol, citral, hinokitiol, eucalyptol, catechol and mixtures thereof. For compositions for contact with the mucosa, or oral compositions, see above.
Nutrients
minerals include calcium, phosphorus, fluoride, zinc, manganese, potassium and mixtures thereof,
vitamins include vit. C and D, thiamine, riboflavin, calcium pantothenate, niacin, folic acid, nicotinamide, pyridoxine, cyanocobalamin, bioflavonoids and mixtures thereof,
aminoacids include but not limited to L-tryptophane, L-lysine, methionine, threonine, levocarnitine, L-carnitine and mixtures thereof.
Antoxidants include those which have been described in previous chapters.

Dentifrices/toothpastes, which are of special importance in oral care applications, often contain:
thickeners/binders from about 0.5% to about 30%,
humectants from about 5% to about 75%,
flavouring agents from about 0.1% to about 5%,
surfactants from about 0.01% to about 6%,
buffering agents from about 0.02% to about 10%,
preservatives from about 0.01% to about 2%,
water deionized from about 4% to about 60%.

Such specific product form needs to provide a good cleaning effect via particulate abrasives and polishing agents such as silicas, aluminas, calcium carbonates, dicalciumphosphates, calcium pyrophosphates, hydroxy apatites, trimethaphosphates, insoluble hexametaphosphates, and in amount between 3% and 60%.

### Opaque Silica-Based Dentifrice

| Ingredients | |
|---|---|
| Sodium Lauryl Sulfate | 1.5-2.5% |
| Sodium Carboxymethyl cellulose or | 0.7-1% |
| Xanthan gum | |
| Sorbitol (70%) | 45% |
| Abrasive silica | 10% |
| Thickening silica | 8-10% |
| PEG 1500 | 5% |
| Titanium Dioxide (opacifier) | 1% |
| Sodium Saccharinate | qs |
| Flavour | <1.5% |
| Aqueous Scleroglucan formulation | 0.1%-5% |
| Colorants | qs |
| Therapeutic agents | qs |
| Preservative | <1% |
| Water deionized | Qsp 100 |

### Translucent or Transparent Dentifrices

often use porous e.g., silica xerogel that possess extremely high cleansing and polishing ability without harmfully abrading the teeth enamel surface, synthetic amorphous complex salts of aluminosilicates (as described by Tamela “Chemistry of the surface and the activity of Alumina-Silica cracking catalyst” Discussions of the Faraday Society No. 8 p 270-279 (1950))

| Ingredients | |
|---|---|
| Sodium N-Lauroyl Sarcosinate | 2% |
| Glycerin | 30% |
| Sorbitol (70%) | 33% |
| Aqueous Scleroglucan formulation | 0.1%-5% |
| Sodium Aluminosilicate | 20% |
| Silicates derivative | 2% |
| Sodium monofluorophosphate | <1% |
| Titanium Dioxide (opacifier) | 1% |
| Sodium Saccharinate | qs |
| Flavour | <2% |
| Stannous fluoride | <0.5% |
| Colorants | qs |
| Preservative | <1% |
| Water deionized | Qsp 100 |

Mouthwashes/mouthrinses are also of importance in oral care applications. They represent aqueous-based formulation where the active ingredients are at lower concentration than toothpastes/dentifrices, and without polishing/abrasive agents and no thickeners.

### Mouthwash 1

| Ingredients | |
|---|---|
| Sodium Saccharinate | 0.05% |
| PEG-40 hydrogenated castor oil | 0.4% |
| Ethanol (96%) | 5.3% |
| Aqueous Scleroglucan formulation | 0.1%-5% |
| Sodium benzoate | 0.25% |
| Flavouring oil | 0.2% |
| Cetylpyridinum Chloride | 0.05% |
| Hydro-glycolic botanical extract | <1% |
| Water deionized | Qsp 100 |

### Mouthwash 2

| Ingredients | |
|---|---|
| Sodium Saccharinate (10%) | 0.8% |
| Menthyl lactate and PPG-26 Buteth-26 | 5% |
| and PEG-40 hydrogenated castor oil | |
| Sorbitol (70%) | 11.5% |
| Chlorhexidine | 0.3% |
| Flavouring oil | <1% |
| Glycerin | 23% |
| Aqueous Scleroglucan formulation | 0.1%-5% |
| Alcohol (95%) | 20% |
| Water deionized | Qsp 100 |

The composition according to the present invention may also be used as a water-based human tissue/personal lubricant such as mucosal lubricant gels, fluids or creams, feminine hygiene products and contain for example:

A] water-soluble thickeners/rheology modifiers (as described in previous chapters)
Most preferable are: modified natural gums such as cellulose derivatives (methylcellulose, hydroxypropyl cellulose, hydroxyethyl cellulose, sodium carboxymethyl cellulose, hydroxyethylmethyl cellulose); natural gums such as arabic, align, guar, tragacanth, pectin, alginic acid and salts, dextran and xanthan gums; synthetic “gums” such as polyvinyl alcohol, polyacrylic acid and polyvinyl pyrrolidone.
B] high molecular weight (MW) polyethylene oxides

Most preferable are ethylene oxides with MW from 900 000 to 4 000 000 in concentration from about 2.5% downward to about 0.5%.

C] humectant polyols that exhibit a very high degree of lubricity and also act as a tissue conditioner such as glycerol, diglycerol, propylene glycol, dipropylene glycol, sorbitol and available poyols made by the hydrogenation of the higher sugars or starches from about 3 to 12% of the lubricating composition.
D] anti-inflammatory agents (as described in previous chapters) and/or healing/soothing agents such as aloe vera, lanolin, allantoin, alpha-bisabolo, panthenol, Nordihydroguaiaretic Acid (NDGA).
E] anti-tack or anti-sticking agents such as polyethoxylated sorbitan monoalkanoates (lauryl, myristyl, palmityl, oleyl, stearyl esters of polyethoxylated sorbitans, where the ethoxyl groups may range from about 15 to 30 in chain length), glycerol monoalkanoates.
F] surfactants and wetting agents (as described in previous chapters)

Most preferable are: anionic surfactants such as sulfates (sodium lauryl sulfate, Zinc coceth sulfate), acylamino acid and salts (sodium cocoyl glutamate, disodium capryloyl glutamate, sodium lauroyl sarcosinate), mild/anti-irritant surfactants such as acylamphoacetates, sulfosuccinates.

G] preservatives (as described in previous chapters)

Most preferable are: propyl and methyl parabens, silver dihydrogenated silver, triclosan. Also of interest are bacteria inhibiting agents (as described in previous chapters), fungicidal and fungistatic agents.

Representative Formulations are Given by the Following Examples;

### Fluid Lubricant Model

| Ingredients | |
|---|---|
| Polyethylene oxides of MW < 4 000 000 | 0.5% to 2.5% |
| Polyethylene oxides of MW < 100 000 | 5% to 10% |
| Humectants | 5% to 10% |
| Aqueous Scleroglucan formulation | 0.1%-5% |
| Preservatives | 0.2% to 1% |
| water-soluble thickeners/rheology | <0.1% |
| modifiers | |
| Water deionized | Qsp 100 |

### Personal Lubricant

| Ingredients | |
|---|---|
| Lubricating agents | 5%-15% |
| Lanolin anhydrous | 8% |
| Sweet almond oil | 5% |
| Coconut oil | 10.75% |
| Cetyl alcohol | 0.5% |
| Mineral oil | 1% |
| Preservative | 0.3% |
| Water deioniz. | Qsp |
| Sodium lauryl sulfate | 1.5% |
| Propylene glycol | 3% |
| Aloe vera gel | 9% |
| Aqueous Scleroglucan formulation | 0.1%-5% |
| Sorbitol | 1% |
| Allantoin | 2.5% |
| Polyethylene oxides of MW < 100 000 | 10%-15% |
| Xanthan gum | 0.5% |
| Coconut flavor | 0.9% |

For feminine hygiene personal cleaning/cleansing products, specific surface active agents that provide solubilizing and detergent properties are necessary such as:

A] Specific anionic surfactants, e.g.
alkanoyl sarcosinates corresponding to the formula RCON(CH3)CH2CH2COOM wherein R is alkyl or alkenyl of about C10 to C20, and M is a water-soluble cation such as ammonium, sodium, potassium and alkanolamine; for example, sodium lauroyl sarcosinate, sodium cocoyl sarcosinate, ammonium lauroyl sarcosinate and sodium myristoyl sarcosinate, TEA salts of sarcosinates;
taurates having between C8 and C16; for example, ammonium, sodium, potassium and alkanolamine salts of lauroyl methyl taurate, myristoyl methyl taurate or cocoyl methyl taurate;
lactylates having between C8 to C16; for example, ammonium, sodium, potassium and alkanolamine salts of lauroyl lactylate, cocoyl lactylate, caproyl lactylate;
glutamates having between C8 to C16; for example, ammonium, sodium, potassium and alkanolamine salts of lauroyl glutamate, myristoyl glutamate, cocoyl glutamate.
   B] Specific amphoteric surfactants, e.g.
betaines which are derived from acylamidopropyl dimethylamine; alkyl betaines, amidoalkyl betaines, phosphobetaines, pyrophosphobetaines, cocamidopropyl betaines and mixtures thereof,
amphocarboxylates,
amidoalkyl sultaines,
amphophosphates,
carboxyalkyl alkyl polyamines,
lauroamphodiacetates.
   C] Specific nonionic surfactants, such as
polyoxyethylene derivatives of polyol esters, wherein the fatty acid part is containing C8 to C22 and the polyol is selected from sorbitol, sorbitan, glucose, α-methyl glucoside, polyglucose, glycerin, pentaerythritol and mixtures thereof. Typical examples are PEG-80 sorbitan laurate, polysorbate 20, PEG-80 sorbitan laurate (PEG stands for polyethyleneglycol).
   Feminine Hygiene and lubricating Lotion

| Ingredients | |
|---|---|
| Lubricating agents | 5%-15% |
| Lanolin anhydrous | 8% |
| Sweet almond oil | 5% |
| Coconut oil | 10.75% |
| Cetyl alcohol | 0.5% |
| Mineral oil | 1% |
| Preservative | 0.3% |
| Water deioniz. | Qsp |
| Sodium lauryl sulfate | 1.5% |
| Propylene glycol | 3% |
| Aqueous Scleroglucan formulation | 0.1%-5% |
| Aloe vera gel | 9% |
| Sorbitol | 1% |
| Allantoin | 2.5% |
| Polyethylene oxides of MW < 100 000 | 10%-15% |
| Xanthan gum | 0.5% |
| Coconut flavor | 0.9% |

### Feminine Hygiene Cleansing Formula

| Ingredients | |
|---|---|
| Chamomile extract | 1.5% |
| Lactic acid (80% solution) | 0.005% |
| Surfactant (as previously described) | 1.5%-10% |
| Solubilizer (as previously described) | 0.5%-1.5% |
| Aqueous Scleroglucan formulation | 0.1%-5% |
| Preservative | 0.2%-1% |
| Water deioniz. | 90%-95% |
| Propylene glycol | 0.8% |
| Fragrance | qs |

The cosmetic or pharmaceutic composition of the invention may also comprise further components which are known to perform a useful function in a cosmetic composition. Examples of such further components include, e.g., UV absorbers such as an oxanilide, a triazine or triazole. The following examples illustrate the effect of scleroglucan which are described and claimed in the invention, or of the combination glucan or scleroglucan with bactericide which are described and claimed in the invention. All percentages are by weight unless otherwise specified. Qsp stands for an amount of carrier (such as water) to be added to reach 100%; qs stands for an amount of ingredient adjustable within a wider range to meet product specifications, e.g. 0-5% b.w., or 0.01-3% b.w.

### Example 1

An aqueous lotion for the treatment of skin roughness is formulated from an aqueous emulsion containing 10% by weight of glyceryl stearate and either 0.005% or 0.05% or 0.5% by weight of β-1,3-scleroglucan (using formulations A-D described further above).

The determination of skin roughness is conducted according to German standard DIN 4768ff. The roughness of the skin is determined by taking silicone-based skin impressions and then measuring the surface profile of the impressions, using computer-aided profilometry. The skin roughness is calculated from the multiple measuring points (100 points per square millimeter). The number of volunteers used is 10. The determination of skin roughness is conducted both prior to application of the test lotion and 8 hours after application of the test lotion. The skin of the forearm is used as the skin test area. For the purpose of comparison, a control experiment is conducted using a placebo containing none of the scleroglucan formulations.

The results obtained show a good reduction of skin roughness (relative to untreated skin) both lotions containing 0.005% by weight or 0.05% by weight of the β-1,3-scleroglucan formulations, while the skin stickiness is reduced in formulations containing the lower amount of scleroglucan.

### Example 2

The skin moisturising activity of the β-1,3-scleroglucan (0.01% or 0.05%) dissolved in water is investigated under defined climatic conditions (22° C. and 60% relative humidity). Test solutions are obtained using formulations A-D, each containing 1% by weight of active, by diluting to obtain aqueous solutions containing 0.05% or 0.01% by weight of the β-1,3-scleroglucan (formulation A), or 0.05% or 0.01% by weight of active mixture (formulations B, C, D).

The test solutions are applied to the forearm. Skin moisture ratings are determined prior to application of the test solution and in regular intervals of 2 hours after application. The determinations are conducted using a Corneometer (model CM820—Courage & Khazuka, Germany). The percentage increase of skin moisture in the treated skin area relative to untreated skin is calculated. The number of volunteers used is 10.

The increase in skin moisture, 1 hour after application of formulation A, is compared to the increase in skin moisture 1 hour after application of formulation B, C or D for each of the concentrations.

Preparations containing formulations B, C and D show an effect superior over the preparation containing formulation A.

### Example 3

Incorporation of 0.05% Scleroglucan (dry weight) into 2.0% of a mixture of glyceryl stearate and PEG-100 stearate (Arlacel® 165), 1.5% cetearyl alcohol (Lanette® O), 1.0% of cetearyl alcohol and Ceteareth-20 (Emulgade® 1000NI), 4.5% Dicaprylyl Ether (Cetiol® OE), 3.0% ethylhexyl stearate (Crodamol® OS), 1,5% C₁₂₋₁₅-alkyl-benzoate (Tegesoft® TN), 1.5% propylene glycol, 0.1% disodium EDTA, 0.8% of a mixture of sodium acrylate copolymer, mineral oil and PPG-1 Trideceth-6 (SALCARE® SC91), 3% of Cyclomethicone (DC 345 fluid), 0.375% lactic acid % ad 100% water increases the “silicone-like” touch by 20% and reduces “waxy” feeling by 15% (average values measured by a panel of 10 trained specialists for psychosensory assessment of topical cosmetic products).

### Example 4

Incorporation of 0.02% Scleroglucan (dry weight) into formulation of 3% acrylates/Beneth-25 methacrylate copolymer (TINOVIS®GTC), 2% Dimethicone Copolyol (Abil® B88183), 0.7% of preservative composition (Germaben® II), 0.25% of 1,2-pentanediol, ad 100% water, increases “silicone-like” touch by 10% and reduces “waxy” feeling by 10% (average values measured by a panel of 10 trained specialists for psychosensory assessment of topical cosmetic products).

### Example 5

Incorporation of 0.02% Scleroglucan (dry weight) into formulation of 3% acrylates/Beneth-25 methacrylate copolymer (TINOVIS®GTC), 2% Dimethicone Copolyol (Abil® B88183), 0.7% of preservative composition (Germaben® II), ad 100% water, increases “silicone-like” touch and reduces “waxy” feeling on skin (determined by a panel of 10 trained specialists for psychosensory assessment of topical cosmetic products).

### EXAMPLE 6

### Preparation of a Toothpaste

The Following Components are Mixed:

| | Formulation: | |
|---|---|---|
| | A | B |
| Ingredients | % by weight | % by weight |
| Distilled water | ad 100 | ad 100 |
| D-glucitol | 40.0 | 40.0 |
| Zeodent 113 | 10.0 | 10.0 |
| glycerol | 20.0 | 20.0 |
| tetrasodium pyrophosphate | 12.0 | 12.0 |
| disodium pyrophosphate | 3.40 | 3.40 |
| sodium lauryl sulfate | 1.37 | 1.37 |
| aromatics | 1.35 | 1.35 |
| PEG-6 | 1.33 | 1.33 |
| sodium carboxymethylcellulose | 1.00 | 1.00 |
| sodium fluoride | 0.50 | 0.50 |
| acrylic acid homopolymer | 0.20 | 0.20 |
| saccharin sodium | 0.20 | 0.20 |
| titanium dioxide | 0.16 | 0.16 |
| P-chitosan | 0.03 | 0.03 |
| and/or Scleroglucan¹⁾ | 0.03 | 0.03 |
| Triclosan | 0.30 | — |
| FD&C Blau CI 42090 (No. 1, 1% sol.) | 0.03 | 0.03 |

¹⁾active agent of Tinocare GL, Ciba Specialty Chemicals

Toothpaste A is distinctly more effective against bacterial plaque than toothpaste B.

### Example 7

### Preparation of a Mouth Wash

The Following Components are Mixed:

Formulation

| | A | B |
|---|---|---|
| | Percent | Percent |
| Ingredients | by weight | by weight |
| Distilled water | ad 100 | ad 100 |
| ethanol | 10.00 | 10.00 |
| glycerol | 10.00 | 10.00 |
| PEO-PPO-PEO block polymer | 2.00 | 2.00 |
| tetrasodium pyrophosphate | 1.50 | 1.50 |
| aromatics | 1.35 | 1.35 |
| disodium pyrophosphate | 0.50 | 0.50 |
| sodium fluoride | 0.50 | 0.50 |
| saccharin sodium | 0.3 | 0.3 |
| P-chitosan²⁾ | 0.02 | 0.02 |
| Scleroglucan¹⁾ | 0.02 | 0.02 |
| Chlorohexidine | 0.2 | — |

¹⁾active agent of Tinocare GL, Ciba Specialty Chemicals
²⁾phosphonochitosan (Tinocare CP, Ciba Specialty Chemicals)

Mouth wash A provides a distinctly better prophylaxis against bacterial plaque than mouth wash B.

### Example 8

### Measurement of the Adsorption and Desorption of Microorganisms

a. Adsorption

Bacteria: S. mutans (ZIB6008); S. mitis (KL-stab.); S. anguinosus (ZIB6006) and S. sanguis (ZIB6010) are plated out anaerobically on BA plates and incubated. One colony each is allowed to grow to a density of about 0.5 OD₆₆₀ in Todd-Hewitt broth as stock solution.

50 mg of hydroxylapatite pearls (HA: Macro-Prep Ceramic Hydroxylapatite, 80 micron, of BioRad) are washed once with 1 ml of sterile H₂O and three times with 1 ml of absorption buffer sterilised by filtration (5 mM KCL, 1 mM CCl₂; 0.1 mM MgCl₂; 1 mM K₂HPO₄, pH 7.2) (cf. Berry & Siragusa (1997); Appl. Environ. Microbiol. 63, 4069-4074). 2 ml of the bacterial solution are centrifuged (10,000 rpm, 5 min) and washed twice with adsorption buffer and are then resuspended in 1 ml of adsorption buffer containing 0.0001-1% of the Scleroglucan used in example 7 and 0.0001%-0.3% of Triclosan and as controls in absorption buffer alone and in absorption buffer containing Glucan or Triclosan only (see table below). These solutions are left for 10 min at 37° C. and then combined with the hydroxylapatite pearls suspended in 1 ml of adsorption buffer and incubated, with slight shaking, for 30 min at 37° C. After the HA pearls have settled, the supernatant is removed and replaced with 0.5 ml absorption buffer containing 0.05% Tween-80. HA pearls are then incubated in the cold and under occasional vortexing. After 15 min 4.5 ml absorption buffer are added, HA-pearls are allowed to sediment and the supernatant is plated accordingly on BA plates and incubated. The results are shown in the following table; compositions of the invention are marked with an asterisk (*).

TABLE a
Adsorption of microorganisms in presence of Scleroglucan (S)
and/or Triclosan (T); No. of colonies

| | | | | |
|---|---|---|---|---|
| agent in liquid | S. mutans | S. mitis | S. anguinosus | S. sanguis |

none
0.0001% S, 0.0001%
T*
0.0002% S
0.0002% T
0.001% S, 0.0003%
T*
0.0013% S
0.0013% T
0.01% S, 0.003% T*
0.013% S
0.013% T
0.1% S, 0.03% T*
0.13% S
0.13% T

Considerably fewer colonies of S. mutans; S. mitis; S. anguinosus; and S. sanguis formed in the dilutions containing both Glucan and Triclosan than in the appropriate dilutions of Glucan or Triclosan alone and even fewer than in the untreated control.

b. Desorption

Bacteria: S. mutans (ZIB6008); S. mitis (KL-stab.); S. anguinosus (ZIB6006) and S. sanguis (ZIB6010) are plated out anaerobically on BA plates and incubated. One colony each is allowed to grow to a density of about 0.5 OD₆₆₀ in Todd-Hewitt broth as stock solution. 50 mg of hydroxylapatite pearls (HA: Macro-Prep Ceramic Hydroxylapatite, 80 micron, of BioRad) are washed once with 1 ml of sterile H₂O and three times with 1 ml of absorption buffer sterilised by filtration (5 mM KCl, 1 mM CCl₂; 0.1 mM MgCl₂; 1 mM K₂HPO₄, pH 7.2) (cf. Berry & Siragusa (1997); Appl. Environ. Microbiol. 63, 4069-4074). 2 ml of the bacterial solution are centrifuged (10,000 rpm, 5 min) and washed twice with adsorption buffer and are then resuspended in 1 ml of adsorption buffer. This solution is combined with the hydroxylapatite pearls suspended in 1 ml of adsorption buffer and incubated, with slight shaking, for 30 min at 37° C. After the HA pearls have settled, the supernatant is removed. The HA pearls are washed once with adsorption buffer and are then incubated, with slight shaking, for 30 min at 37° C. with 1 ml of adsorption buffer containing 0.0001-1% of the Scleroglucan used in example 7 and 0.0001%-0.3% of Triclosan and as controls Glucan or Triclosan alone and absorption buffer alone (see table b below).

After the HA pearls have settled, the supernatant is removed and replaced with 0.5 ml absorption buffer containing 0.05% Tween-80. HA pearls are then incubated in the cold and under occasional vortexing. After 15 min 4.5 ml absorption buffer are added, HA-pearls are allowed to settle and the supernatant is plated accordingly on BA plates for quantification. The results are shown in the following table; compositions of the invention are marked with an asterisk (*).

TABLE b
Desorption of microorganisms in presence of Scleroglucan (S)
and/or Triclosan (T); No. of colonies

| | | | | |
|---|---|---|---|---|
| agent in liquid | S. mutans | S. mitis | S. anguinosus | S. sanguis |

none
0.0001% S, 0.0001%
T*
0.0002% S
0.0002% T
0.001% S, 0.0003%
T*
0.0013% S
0.0013% T
0.01% S, 0.003% T*
0.013% S
0.013% T
0.1% S, 0.03% T*
0.13% S
0.13% T

Considerably fewer colonies of any formed when combining Glucan and Triclosan than in the appropriate dilutions of Glucan or Triclosan alone and even fewer than in the untreated control.

### Example 9

Bacteria: S. mutans (ZIB6008); S. mitis (KL-stab.); S. anguinosus (ZIB6006), S. sobrinus (OMZ 176) and S. sanguis (ZIB6010) are plated out on BA plates. Stock solutions are grown in Todd-Hewitt broth at 37° C. from overnight single colony cultures.

Bacteria stock solutions are sonicated for 15 sec at 30 W, centrifuged for 5 min at 8000 rpm, the pellet then re-suspended in PBS, again centrifuged and re-suspended in a sterilized 1:1 mixture of culture medium and human saliva or in PBS (phosphate buffered saline, pH 7.2-7.4) and media to yield a final concentration of 10⁸-10⁹/ml bacteria. A part is plated for CFU (colony forming units) determination.

### a) Influence on Planctonic Cultures

The rest of this solution is either used alone or mixed with different amounts of Scleroglucan (SC) or Triclosan (TR) and mixtures thereof as indicated in Tables 9 or as a control with the appropriate volumes of PBS only. After 2 min at 37° C. bacteria are centrifuged (5 min at 8000 rpm), the pellet washed by PBS, re-centrifuged and suspended in a sterilized mixture of culture medium/human saliva 1:1 or with medium and PBS to yield the test (TST) solution or the control solution (CTRL). Parts of TST and CTRL-solutions are plated and the rests are added to borosilicate glass plates mounted in flow-chambers essentially as described by Decker et al J. Period Res 40: 373 (2005) and incubated for 60 min at 37° C. Test plates are shortly washed in water and densities and cell vitality of adsorbed cells determined accordingly (see Decker et al 2005).

### b) Influence on Attached Cells

Plates incubated for 60 min with CTRL cultures as described above are removed, shortly washed in PBS and then immersed for 2 min at 37° C. into a mixture of PBS containing SC, TR or mixtures of SC/TR as described in Tables 9. Then plates are shortly washed in water and processed for density and vitality assessment as described above.

### c) Results

In the planctonic phase the combinations of SC/TR show the best antibacterial effect as measured by CFU reduction.

Adhesion of vital cells to glass plates is lowest when pre-treating bacteria in the planctonic phase with combinations of SC/TR.

After treatment of the biofilm the amount of vital cells is lowest when using combinations of SC/TR.

TABLE 9.1
Influence on planctonic phase Scleroglucan (S) and/or
Triclosan (T); No. of colonies

| agent in | | | S. | S. | |
|---|---|---|---|---|---|
| liquid | S. mutans, | S. mitis, | anguinosus, | sanguis | S. sobrinus |
| none | | | | | |
| 0.0001% S, | | | | | |
| 0.0001% T* | | | | | |
| 0.0002% S | | | | | |
| 0.0002% T | | | | | |
| 0.001% S, | | | | | |
| 0.0003% T* | | | | | |
| 0.0013% S | | | | | |
| 0.0013% T | | | | | |
| 0.01% S, | | | | | |
| 0.003% T* | | | | | |
| 0.013% S | | | | | |
| 0.013% T | | | | | |
| 0.1% S, | | | | | |
| 0.03% T* | | | | | |
| 0.13% S | | | | | |
| 0.13% T | | | | | |

In the planctonic phase the combinations of SC/TR show the best antibacterial effect as measured by CFU reduction.

TABLE 9.2
Influence on cell adsorbtion when treated in planctonic phase;
Scleroglucan (SC) and/or Triclosan (TR); % cell density and %
cell viability of untreated control

| agent in | | | S. | S. | |
|---|---|---|---|---|---|
| liquid | S. mutans, | S. mitis, | anguinosus, | sanguis | S. sobrinus |
| none | | | | | |
| 0.0001% S, | | | | | |
| 0.0001% T* | | | | | |
| 0.0002% S | | | | | |
| 0.0002% T | | | | | |
| 0.001% S, | | | | | |
| 0.0003% T* | | | | | |
| 0.0013% S | | | | | |
| 0.0013% T | | | | | |
| 0.01% S, | | | | | |
| 0.003% T* | | | | | |
| 0.013% S | | | | | |
| 0.013% T | | | | | |
| 0.1% S, | | | | | |
| 0.03% T* | | | | | |
| 0.13% S | | | | | |
| 0.13% T | | | | | |

The relative amount of vital cells adsorbing to glass is lowest when using combinations of SC/TR.

TABLE 9.3
Influence on cell desorption when treating bio-film of adsorbed
bacteria with Scleroglucan (SC) and/or Triclosan (TR); % cell
density and % cell viability of untreated control

| agent in | | | S. | S. | |
|---|---|---|---|---|---|
| liquid | S. mutans | S. mitis | anguinosus | sanguis | S. sobrinus |
| none | | | | | |
| 0.0001% S, | | | | | |
| 0.0001% T* | | | | | |
| 0.0002% S | | | | | |
| 0.0002% T | | | | | |
| 0.001% S, | | | | | |
| 0.0003% T* | | | | | |
| 0.0013% S | | | | | |
| 0.0013% T | | | | | |
| 0.01% S, | | | | | |
| 0.003% T* | | | | | |
| 0.013% S | | | | | |
| 0.013% T | | | | | |
| 0.1% S, | | | | | |
| 0.03% T* | | | | | |
| 0.13% S | | | | | |
| 0.13% T | | | | | |

After treatment of the biofilm the relative amount of vital cells is lowest when using combinations of SC/TR.

1. An antibacterial composition for contact with the mucosa or other tissues of the oral cavity, comprising
a) a glucan, in an amount of 0.001 to 0.2%, relative to the weight of the total composition, and
b) a bactericide selected from the group consisting of benzoic acid, its salts and esters; propionic acid and its salts; salicylic acid and its salts; sorbic acid and its salts; formaldehyde; paraformaldehyde; o-phenylphenol and its salts; inorganic sulphites and hydrogen sulphites; sodium iodate; chlorobutanol; 4-hydroxybenzoic acid and its salts and esters; 3-acetyl-6-methylpyran-2,4(3H)-dione; formic acid; sodium formiate; dibromohexamidine and its salts; undec-10-enoic acid and salts; hexetidine; 5-bromo-5-nitro-1,3-dioxane; bronopol; 2,4-dichlorobenzyl alcohol; triclocarban; 2.4.4′-trichloro-2′-hydroxy-diphenylether (Triclosan); 4-chloro-3,5-xylenol; imidazolidinyl urea; poly(1-hexamethylenebiguanide hydrochloride); 2-phenoxyethanol; hexamethylenetetramine; methenamine 3-chloroallylochloride; 1-(4-chlorophenoxy)-1-(imidazol-1-yl)-3,3-dimethylbutan-2-one; 1,3-bis(hydroxymethyl)-5,5-dimethylimidazolidine-2,4-dione; benzyl alcohol; 1-hydroxy-4-methyl-6(2,4,4-trimethylpentyl)-2-pyridon or its monoethanolamine salt; methyldibromoglutaronitrile; bromochlorophen; 4-isopropyl-m-cresol; mixture of 5-Chloro-2-methyl-isothiazol-3(2H)-one and 2-methylisothiazol-3(2H)-one with magnesium chloride and magnesium nitrate; clorophene; 2-chloroacetamide; chlorhexidine and its digluconate, diacetate and/or dihydrochloride; 1-phenoxypropan-2-ol; alkyl (C₁₂-C₂₂) trimethyl ammonium bromide and/or chloride; 4,4-dimethyl-1,3-oxizalidine; N-(hydroxymethyl)-N-(dihydroxymethyl-1,3-dioxo-2,5-imidazolidinyl-4)-N′-(hydroxymethyl)urea; hexamidine and its salts; glutaraldehyde; chlorphenesin; sodium hydroxymethylglycinate; benzethonium chloride; benzalkonium chloride, bromide and/or saccharinate; benzylhemiformal, listerine, alexidine, and essential oils including thymol, geraniol, carvacrol, citral, hinokitiol, eucalyptol, eugenol, menthol, or catechol.

2. A composition comprising
a) 0.001 to 0.2% by weight, relative to the weight of the total composition, of a scleroglucan of mean molecular weight one million to twelve million;
b) a cosmetically or pharmaceutically acceptable carrier; and
c) a further component selected from the group consisting of lactic acid, lactate and 1,2-pentanediol,
in an amount of 0.005 to 3% by weight, relative to the weight of the total composition.

3. A composition according to claim 2 comprising
a) 0.005 to less than 0.05% by weight, relative to the weight of the total composition, of a scleroglucan of mean molecular weight one million to twelve million; and
b) a cosmetically or pharmaceutically acceptable carrier.

4. A composition according to claim 1 comprising a bactericide as defined as component (b) of claim 1, and wherein the weight ratio bactericide:glucan component (a) is from the range 1:10-10:1.

5. A composition according to claim 2 wherein said composition is in the form of a cosmetical or dermatological skin composition, containing as component d) a cosmetically or dermatologically acceptable carrier.

6. A composition according to claim 1 wherein said composition is an oral care composition, a feminine hygiene composition, a composition for the treatment of a medical implant a composition for the treatment of an oral implant, an eye drop formulation, an eye make-up composition or an eye make-up remover composition.

7. An oral care composition according to claim 6, wherein said composition is a tooth paste or gel, a mouth wash, a gargle, an inhalant, a denture or implant or brace or a cleaner thereof, or an adhesive paste.

8. An oral care composition according to claim 7, additionally containing a further agent suitable to inhibit alkaline phosphatase, wherein said agent is selected from the group consisting of polyanionic polysaccharides, polyanionically-derivatised polysaccharides, phosphochitosanes and phosphochitosanes.

9. A composition according to claim 1, wherein the bactericide of component b) is selected from the group consisting of Triclosan, chlorohexidin, hexetidine, and listerine.

10. A composition according to claim 2, characterized in that component (a) is a scleroglucan having a three-dimensional crosslinked triple helix structure, having the structural formula 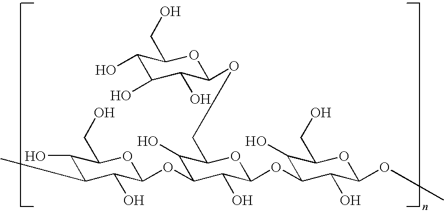
wherein n is a number which provides the scleroglucan component with a mean molecular weight of one million to twelve million.

11. A composition according to claim 2, wherein component (a) is a scleroglucan which is obtained by cultivation of microorganisms, in the form of the plant-pathogenic fungi imperfecti Sclerotium rolfsii.

12. A composition according to claim 5 wherein said composition is a skin care composition, shampoo and/or hair conditioner composition in the form of a gel or viscous liquid.

13. A composition according to claim 2 wherein said composition is in the from of an aqueous lotion, a water-in-oil or an oil-in-water emulsion, an oil or oil-alcohol lotion, a vesicular dispersion of an anionic or nonionic amphiphilic lipid, an aqueous, aqueous-alcohol, alcohol or oil-alcohol gel, a solid stick or an aerosol.

14. A composition according to claim 13 wherein said composition is a water-in-oil emulsion or said oil-in-water emulsion, the cosmetically acceptable carrier of component b) comprises 5 to 50% of an oil phase and 47 to 94.95% of water, each based on the total weight of the composition, and optionally a further component acting as an emulsifier and/or surfactant.

15. A composition according to claim 1 wherein said composition additionally comprises at least one substance selected from the group consisting of antiphlogistic agents, antiinflammatory agents, vitamins, antioxidants, antipsoriatic agents, further skin actives, cell proliferation regulators, antiallergic, UV protecting, moisturizing, antiaging agents, DNA-protectants, emollients, additional thickening agents, moisture-retention agents, film formers, preservatives, perfumes and colourants.

16. A composition according to claim 12, wherein said composition is an anti-inflammatory skin care preparation, a sunscreen lotion, an after-sun skin care preparation, a revitalizing skin care preparation, or an anti-aging skin care preparation.

17. A composition according to claim 1 containing the scleroglucan in an amount between 0.05 and 0.1% by weight of the total composition.

18. A composition according to claim 8 formulated as a microemulsion or dispersion of nano-scaled particles.

19. (canceled)

20. A process for the preparation of a cosmetical or dermatological formulation for the treatment or prevention of inflammatory or allergic conditions, skin-aging, skin irritation or inflammation by sun exposure, wrinkles, or for wound healing, wherein said process comprises
adding 0.005 to less than 0.05% by weight, relative to the weight of the total formulation, of a scleroglucan of mean molecular weight one million to twelve million together with lactic acid, a lactate and/or 1,2-pentanediol, to a cosmetically or dermatologically acceptable carrier and mixing thoroughly.

21. A concentrate for the preparation of a cosmetic or pharmaceutical formulation, wherein said concentrate comprises
a) 0.3 to 3.0% by weight, relative to the total weight of the concentrate, of a scleroglucan of mean molecular weight one million to twelve million;
b) a cosmetically or pharmaceutically acceptable aqueous carrier, and
c) lactic acid or a lactate and/or 1,2-pentanediol in a weight ratio relative to component (a) from the range 2:1 to 20:1.

22. A method for reducing the number of bacteria and/or minimise adhesion of bacteria in mucosal or oral environments which method comprises contacting a composition according to claim 1 with the mucosa or oral tissue.

23. A composition according to claim 2 comprising
a) 0.001 to 0.1% by weight, relative to the weight of the total composition, of a scleroglucan of mean molecular weight one million to twelve million;
b) a cosmetically or pharmaceutically acceptable carrier; and
c) a further component selected from the group consisting of lactic acid, lactate and 1,2-pentanediol, in an amount of 0.02 to 1.0% by weight, relative to the weight of the total composition.

24. A composition according to claim 23 comprising
a) 0.005 to less than 0.05% by weight, relative to the weight of the total composition, of a scleroglucan of mean molecular weight one million to twelve million;
b) a cosmetically or pharmaceutically acceptable carrier; and
c) a further component selected from the group consisting of lactic acid, lactate and 1,2-pentanediol, in an amount of 0.02 to 1.0% by weight, relative to the weight of the total composition.

25. A composition according to claim 6 wherein said composition is in the form of an aqueous paste or gel or an aqueous liquid.

26. A process according to claim 20 for the preparation of a cosmetic or dermatological formulation for the treatment or prevention of inflammatory or allergic conditions, skin-aging, skin irritation or inflammation by sun exposure, wrinkles, or for wound healing, wherein said process comprises
adding 0.001 to 0.2% by weight, relative to the weight of the total formulation, of a beta-1,3-scleroglucan of mean molecular weight one million to twelve million, together with lactic acid, a lactate and/or 1,2-pentanediol, to a cosmetically or dermatologically acceptable carrier and mixing thoroughly.
